(19) United States
(12) Patent Application Publication
(10) Pub. No.: US 2004/0241168 A1
(43) Pub. Date: Dec. 2, 2004

(54) Compositions and methods for the treatment and clinical remission of psoriasis

(76) Inventor: Jose A. O'Daly, Florham Park, NJ (US)

Correspondence Address: BOND, SCHOENECK & KING, PLLC, ONE LINCOLN CENTER, SYRACUSE, NY, 13202-1355, US

(21) Appl. No.: 10/774,928

(22) Filed: Feb. 9, 2004

Related U.S. Application Data
(63) Continuation-in-part of application No. 10/687,892, filed on Oct. 17, 2003.
Application No. 10/687,892 is a continuation of application No. 09/809,003, filed on Mar. 16, 2001, now Pat. No. 6,673,351.

(51) Int. Cl. A61K 39/395

(52) U.S. Cl. 424/154.1

(57) ABSTRACT

A treatment for psoriasis and related maladies has a mechanism of action that includes an inhibition or blockade of T cell rolling by interference with the CLA-E selectin interaction and interference of endothelial binding or diapadesis by induced by blocking the LFA-1/ICAM interaction and/or the VLA/VCAM interaction with endothelial cells.

This application is a continuation-in-part of U.S. application Ser. No. 10/687,892 which was on filed Oct. 17, 2003 which is a continuation of U.S. patent application Ser. No. 09/809,003 which issued as U.S. Pat. No. 6,673,351 which issued on Jan. 6, 2004.

### FIELD OF THE INVENTION

The present invention relates generally to immunotherapeutic agents or therapeutic agents, compositions comprising those agents, and methods of use of those agents and compositions for the treatment and clinical remission of psoriasis.

### BACKGROUND

Psoriasis is a chronic, genetically-influenced, remitting and relapsing scaly and inflammatory skin disorder of unknown etiology that affects 1 to 3 percent of the world's population. There are several types of psoriasis, including plaque, pustular, guttate and arthritic variants. As reported by Stephanie Mehlis and Kenneth Gordon, the immunology of psoriasis has been studied and it appears that the mechanism of the human immune system that triggers symptoms of psoriasis is closely tied to a lymphatic infiltrate that consists T-cell lymphocytes. Journal of the American Academy of Dermatology, 2003;49:S44-50. T cells play a role in the initiation and maintenance of psoriasis. The role of T cells in the initiation and maintenance of psoriasis can be broken down into three areas: (1) the initial activation of T cells, (2) the migration of T cells into the skin, and (3) the effector function of the T cells in the skin by the secretion of cytokines and the magnification of the immunologic cascade.

The initial activation of a T cell requires three steps. The first step is binding: the T Cell becomes momentarily and reversibly attached to an antigen-presenting cell (APC). This process is mediated through surface molecules used for adhesion including leukocyte function associated antigen (LFA)-1 and CD2 on the T cells and intercellular adhesion molecule (ICAM)-1 and LFA-3 on the APC. The next step is an antigen-specific activation process called signal 1. Here, the T cell's specific T-cell receptor recognizes an antigen presented on the major histocompatibility complex (MHC I or II) by the APC. The final step is a non-antigen specific cell-cell interaction referred to as signal 2 or co-stimulation. If co-stimulation does not occur, the T cell will not respond and will either undergo apoptosis or be rendered unresponsive in the future, a process called anergy.

Just as T cells must become activated to induce or maintain psoriasis, so must they be present in the skin. The process of T cells migrating or “trafficking” to the skin is also a multi-step process regulated by secreted factors and cell-cell interactions between the T cell and the endothelium. An activated T cell in the circulation must be slowed and then bound to the endothelium before migrating into the affected tissue, in this case, the skin. The first step in this process, rolling, is mediated by cell-cell interactions such as cutaneous lymphocyte antigen (CLA) on the migrating T cell and E-selectin on the endothelial cell. Rolling slows the cells down so they may bind to the blood vessel walls and become immobile. There are multiple requirements for binding, including the activation of surface proteins on the T cells, mediated by small chemotactic proteins called chemokines, and T cell endothelial surface protein binding including LFA-1/ICAM and VLA/VACM interactions. Once this binding step has occurred, the T cell may migrate through the blood vessel wall in a process called diapedesis, and participate in the local immune response in psoriasis.

The final step in the immunologic process of psoriasis is the induction of the keratinocyte changes by T cells and secretions of other inflammatory cells. This step can involve many cell types, including T cells, local macrophages, dendritic cells, vascular endothelium, and even keratinocytes. Though there are many potential interactions between these cell types that could have a profound influence on psoriasis, it is likely that a cascade of cytokines, secreted by many different cells in the local environment of the psoriatic plaque, plays a central role in the phenotypic responses in psoriasis (Table I). Importantly, both CD4(+) and CD8(+) T cells produce T1 type cytokines, ie, interferon-γ (IFN-γ), and IL-2. These cytokines influence other cells locally to secrete a plethora of proteins including chemokines, tumor necrosis factor-α (TNF-α), granulocyte-macrophage colony stimulating factor (GM-CSF), epidermal growth factor (EGF), and IL-8. These regulate the migration of new inflammatory cells into the skin and increase the activity of these cells and keratinocytes, resulting in a psoriatic plaque. There is a need to provide methods and compositions to treat psoriasis and other maladies that are related to T-cell lymphocytes infiltrating certain membranes.

### SUMMARY OF THE INVENTION

A treatment for psoriasis and related maladies has a mechanism of action that includes an inhibition or blockade of T cell rolling by interference with the CLA-E selectin interaction by a novel cytokine and interference of endothelial binding or diapadesis by a novel cytokine induced by stimulation of an unknown T cell clone that blocks the LFA-1/ICAM interaction and/or the VLA/VCAM interaction with endothelial cells.

### DETAILED DESCRIPTION OF THE INVENTION

The present invention concerns novel compositions and methods for the treatment and clinical remission of psoriasis. The preferred embodiment is represented by compositions which comprise immunogenic polypeptides or the nucleic acids encoding them. In one embodiment of the invention, the subject polypeptides can be isolated from Leishmania protozoa and, preferably, from killed Leishmania amastigote protozoa. The polypeptides of the subject invention can be obtained from protozoa of the Leishmania genus using standard protein isolation procedures which are known in the art. Also contemplated by the present invention are immunotherapeutic agents and pharmaceutical compositions incorporating the immunogenic polypeptides of the present invention. In one embodiment, a first-generation polyvalent immunotherapeutic agent is provided, comprising a polypeptide isolate of a mixture of a plurality of Leishmania species, such as L. (L)amazonensis, L. (L)venezuelensis, L. (L)brasiliensis, L. (L)chagasi, L. (L) donovani, L. (L)infantum, L.(L)major, L.(L)panamensis, L. (L)tropica, and L. (L) guyanensis. Preferably, the mixture comprises L.(L)amazonensis, L.(L)venezuelensis, L.(V)brasiliensis, and L. (L)chagasi. Most preferably, the mixture consists of these four species. The organisms are preferably cultivated in the amastigote stage in the synthetic culture medium specified in Table 1, supplemented with 5% fetal bovine serum, typically at about 30-34° C. Subsequently, and during the stationary phase of growth, the amastigotes are subjected to a medium containing an amount of N-p-tosyl-L-Lysine chloromethyl ketone (TLCK) or a pharmacologically acceptable salt thereof effective to kill the cells. The dead cells are then isolated and treated with the non-ionic detergent Nonidet p-40 (NP40) to solubilize the surface antigens, which are discarded. The particulate antigens that comprise the immunogenic polypeptides of the present invention can be collected by centrifugation following cell disruption. These polypeptides are washed with phosphate-buffered saline (PBS) and subsequently resuspended by sonication for 5 minutes at 4° C. in PBS containing alumina.

In another embodiment, a first-generation monovalent immunotherapeutic agent is described, comprising a polypeptide isolate of a single Leishmania species chosen from the group consisting of L. (L)amazonensis, L. (L)venezuelensis, L. (V)brasiliensis, L. (L) chagasi, L. (L)donovani, L. (L)infantum, L. (L)major, L. (L)panamensis, L. (L)tropica, and L. (L) guyanensis. Preferably, the single Leishmania species is chosen from the group consisting of L. (L)amazonensis, L. (L)venezuelensis, L. (V)brasiliensis, and L. (L)chagasi. Procedures for the preparation of this immunotherapeutic agent are otherwise identical to those disclosed above for the first-generation polyvalent immunotherapeutic agent.

In another embodiment, a second-generation polyvalent immunotherapeutic agent is described, comprising a polypeptide isolate of a mixture of a plurality of Leishmania species, such as L. (L)amazonensis, L. (L)venezuelensis, L. (V)brasiliensis, L. (L) chagasi, L. (L)donovani, L. (L)infantum, L. (L)major, L. (L)panamensis, L. (L)tropica, and L.(L) guyanensis. Preferably, the mixture comprises L.(L)amazonensis, L.(L)venezuelensis, L.(V)brasiliensis, and L. (L)chagasi. Most preferably, the mixture consists of these four species. The organisms are preferably cultivated in the amastigote stage in the synthetic culture medium specified in Table 1, supplemented with 5% fetal bovine serum, typically at about 30-34° C. Subsequently, and during the stationary phase of growth, the amastigotes are subjected to a medium containing an amount of N-p-tosyl-L-Lysine chloromethyl ketone (TLCK) or a pharmacologically acceptable salt thereof effective to kill the cells. The dead cells are then isolated and treated with the non-ionic detergent Nonidet p-40 (NP40) to solubilize the surface antigens, which are discarded. The particulate antigens that comprise the immunogenic polypeptides of the present invention can be collected by centrifugation following cell disruption. These polypeptides are washed with phosphate-buffered saline (PBS) and subsequently resuspended by sonication for 5 minutes at 4° C. in 8 M Urea, 0.025 M Tris (Tris-hydroxy-methyl-amino-methane). The polypeptides are then subjected to chromatography on a DEAE-Sephadex column with a stepwise elution from 0.05-0.3 M NaCl in a solution containing 8 M Urea, .025 M Tris, pH 8.3. Seven protein fractions are collected, and an inoculum comprising each protein fraction is made by resuspending the polypeptides of each fraction in PBS containing alumina.

In another embodiment, a second-generation monovalent immunotherapeutic agent is described, comprising a polypeptide isolate of a single Leishmania species chosen from the group consisting of L. (L)amazonensis, L. (L)venezuelensis, L. (V)brasiliensis, L.(L)chagasi, L.(L)donovani, L.(L)infantum, L.(L)major, L.(L)panamensis, L. (L)tropica, and L. (L)guyanensis. Preferably, the single Leishmania species is chosen from the group consisting of L. (L)amazonensis, L. (L)venezuelensis, L. (V)brasiliensis, and L. (L) chagasi. Procedures for the preparation of this immunotherapeutic agent are otherwise identical to those disclosed above for the second-generation polyvalent immunotherapeutic agent.

Alternatively, the subject polypeptides can be synthesized according to known procedures and techniques, or produced recombinantly by transforming a host cell with one or more of the nucleotide sequences encoding the desired polypeptides. The polypeptides can be expressed in the host cell such that they can be isolated and purified to a desired degree of purification. The subject polypeptides can be used in accordance with the subject invention as a third-generation immunotherapeutic agent to treat psoriasis.

The instant invention further concerns nucleic acid sequences that can be useful in transforming appropriate host cells to cause them to produce the polypeptides of the invention; in administration to a warm-blooded animal, either directly or as part of a pharmaceutically-acceptable composition, to generate an immune response and thereby induce clinical remission of psoriasis in the animal; as labelled probes for genetic analysis; or as nucleic acid molecular weight markers.

One of ordinary skill in the art of molecular biology can obtain nucleic acids encoding the polypeptides of the present invention in view of the teachings provided herein. For example, the polypeptides of the first-generation immunotherapeutic agent of the present invention have been isolated and purified from protozoa of the Leishmania genus and comprise eight bands, identified by SDS-PAGE, representing eight distinct polypeptides having apparent molecular weights of 21, 33, 44, 50, 55, 58, 65, and 77 kDa, respectively. Each of these bands represents a separate polypeptide that can be isolated and sequenced in accordance with standard amino acid sequencing procedures. The polypeptides of each second-generation immunotherapeutic agent were purified by subjecting the first-generation immunotherapeutic agent containing the mixture of eight polypeptides to chromatography on diethylaminoethyl(DEAE)-Sephadex. Two fractions having all the activity to cure psoriasis were isolated and totally reduced and alkylated by standard procedures. These fractions were subjected to electrophoresis on acrylamide gels to separate the constituent polypeptides, and the amino acid sequence of each polypeptide was obtained by standard protein sequencing procedures. The nucleotide sequences encoding each of these polypeptides can be derived from these amino acid sequences by application of the genetic code.

Additionally, the present invention contemplates the production of large quantities of the immunogenic polypeptides of the invention via introduction of the nucleic acids encoding them to microbial host cells. The nucleic acids can be introduced directly into the genome of the host cell or can first be incorporated into a vector which is then introduced into the host. Exemplary methods of direct incorporation include transduction by recombinant phage or cosmids, transfection where specially treated host bacterial cells can be caused to take up naked phage chromosomes, and transformation by calcium precipitation. These methods are well known in the art.

Exemplary vectors include plasmids, cosmids, and phages. A genomic library for a Leishmania species can be created by routine means, and DNA of interest isolated therefrom. For example, DNA of Leishmania protozoa can be isolated and restricted with known restriction enzymes. The resulting DNA fragments can then be inserted into suitable cloning vectors for introduction to a compatible host. Depending on the contemplated host, the vector may include various regulatory and other regions, usually including an origin of replication, one or more promoter regions, and markers for the selection of transformants. In general, the vectors will provide regulatory signals for expression and amplification of the DNA of interest.

Various markers may be employed for the selection of transformants, including biocide resistance, particularly to antibiotics such as ampicillin, tetracycline, trimethoprim, chloramphenicol, and penicillin; toxins, such as colicin; and heavy metals, such as mercuric salts. Alternatively, complementation providing an essential nutrient to an auxotrophic host may be employed.

Hosts which may be employed according to techniques well known in the art for the production of the polypeptides of the present invention include unicellular microorganisms, such as prokaryotes, i.e., bacteria; and eukaryotes, such as fungi, including yeasts, algae, protozoa, molds, and the like, as well as plant cells, both in culture or in planta. Specific bacteria which are susceptible to transformation include members of the Enterobacteriaceae, such as strains of Escherichia coli; Salmonella; Bacillaceae, such as Bacillus subtilis; Pneumococcus; Streptococcus; Haemophilus influenzae, and yeasts such as Saccharomyces, among others. As used herein, the term microbial host cell encompasses all of these prokaryotic and eukaryotic organisms, including plant cells, both in culture and in planta.

Universal probes can be obtained which hybridize with certain of the fragments of a DNA library, allowing identification and selection (or “probing out”) of the genes of interest, i.e., those nucleotide sequences which encode the polypeptides described as part of the present invention. The isolation of these genes can be performed using techniques which are well known in the art of molecular biology. The isolated genes can be inserted into appropriate vectors for use in the transformation of microbial host cells. In addition, these genes can be subjected to standard nucleic acid sequencing procedures to provide specific information about the nucleotide sequence of the genes encoding the subject polypeptides.

It is now well known in the art that when synthesizing a gene for improved expression in a host cell it is desirable to design the gene such that its frequency of codon usage approaches the frequency of preferred codon usage of the host cell. For purposes of the subject invention, “frequency of preferred codon usage” refers to the preference exhibited by a specific host cell in usage of nucleotide codons to specify a given amino acid. To determine the frequency of usage of a particular codon in a gene, the number of occurrences of that codon in the gene is divided by the total number of occurrences of all codons specifying the same amino acid in the gene. Similarly, the frequency of preferred codon usage exhibited by a host cell can be calculated by averaging frequency of preferred codon usage in a large number of genes expressed by the host cell. It is preferable that this analysis be limited to genes that are highly expressed by the host cell.

Thus, in one embodiment of the subject invention, bacteria, plants, or other cells can be genetically engineered, e.g., transformed with genes from protozoa of the Leishmania spp., to attain desired expression levels of the subject polypeptides or proteins. To provide genes having enhanced expression, the DNA sequence of the gene can be modified to comprise codons preferred by highly expressed genes to attain an A+T content in nucleotide base composition which is substantially that found in the transformed host cell. It is also preferable to form an initiation sequence optimal for said host cell, and to eliminate sequences that cause destabilization, inappropriate polyadenylation, degradation and termination of RNA and to avoid sequences that constitute secondary structure hairpins and RNA splice sites. For example, in synthetic genes, the codons used to specify a given amino acid can be selected with regard to the distribution frequency of codon usage employed in highly expressed genes in the host cell to specify that amino acid. As is appreciated by those skilled in the art, the distribution frequency of codon usage utilized in the synthetic gene is a determinant of the level of expression.

Assembly of the genes of this invention can be performed using standard technology known in the art. A structural gene designed for enhanced expression in a host cell can be enzymatically assembled within a DNA vector from chemically synthesized oligonucleotide duplex segments. The gene can then be introduced into the host cell and expressed by means known in the art. Preferably, the protein produced upon expression of the synthetic gene is functionally equivalent to a native protein. According to the subject invention, “functionally equivalent” refers to identity or near identity of function. A synthetic gene product which has at least one property relating to its activity or function that is similar or identical to a natural protein is considered functionally equivalent thereto.

It is also well known in the art that the nucleotide sequences of the subject invention can be truncated such that certain of the resulting fragments of the original full-length sequence can retain the desired characteristics of the full-length sequence. A wide variety of restriction enzymes are well known by those skilled in the art to be suitable for generating fragments from larger nucleic acid molecules. For example, it is well known that Bal31 exonuclease can be conveniently used for time-controlled limited digestion of DNA. See, for example, Maniatis et al. (1982) Molecular Cloning: A Laboratory Manual, Cold Spring Harbor Laboratory, New York, pages 135-139. See also Wei et al. (1983) J Biol. Chem. 258:13006-13512. Thus, Bal31 exonuclease (commonly referred to as “erase-a-base” procedures) allows for the removal of nucleotides from either or both ends of the subject nucleic acids, consequently generating a wide spectrum of fragments, many of which encode products that are functionally equivalent to the natural polypeptide sequences of the present invention. Labeling procedures are also well known, and the ordinarily skilled artisan could routinely screen the labeled fragments for their hybridization characteristics to determine their utility as probes. For example, it is routine to label nucleic acids for use as specific and selective probes in genetic identification or diagnostic procedures. A person of ordinary skill in the art would recognize that variations or fragments of those sequences, which specifically and selectively hybridize to the DNA of Leishmania spp., could also function as a probe. It is within the ordinary skill of persons in the art, and does not require undue experimentation, to determine whether a segment of the subject nucleic acids is a fragment or variant which specifically and selectively hybridizes in accordance with the subject invention. Therefore, fragments or variants of these nucleic acids can be useful as probes to identify, diagnose, or distinguish Leishmania species.

It would also be recognized that the polynucleotides or peptides of the subject invention can be useful as molecular weight markers in respective nucleic acid or amino acid molecular weight determinations or assays.

In order to obtain a first-generation immunotherapeutic agent according to the subject invention, organisms of the genus Leishmania can be cultivated in synthetic culture medium comprising the ingredients listed in Table 1. In a preferred embodiment, the culture medium is supplemented with 5% fetal bovine serum. Cultivation of the protozoa according to the subject invention is typically carried out at about 30-34° C. In a particularly preferred embodiment, cultivation of the protozoa is carried out in the amastigote stage of its life cycle.

TABLE 1
Leishmania culture medium.

| Ingredient | mg/lt |
|---|---|
| Methionine | 140 |
| Tryptophan | 50 |
| α-Amino Adipic Acid | 3 |
| Asparagine | 165 |
| Cystine | 47 |
| Histidine | 6 |
| Aspartic Acid | 120 |
| Alanine | 512 |
| Proline | 248 |
| Lysine | 337 |
| Taurine | 6 |
| Isoleucine | 191 |
| Ornithine | 3 |
| Tyrosine | 210 |
| β-alanine | 80 |
| Phosphoserine | 23 |
| α-amino Butyric Acid | 8 |
| Leucine | 440 |
| Arginine | 413 |
| Serine | 220 |
| Hydroxylysine | 12 |
| Glutamine | 164 |
| Glutamic Acid | 420 |
| Cysteine | 0.5 |
| Phosphoethanolamine | 25 |
| Threonine | 200 |
| Glycine | 235 |
| Phenylalanine | 240 |
| Valine | 266 |
| d-Pantothenic Acid | 1 |
| Ascorbic Acid | 0.05 |
| p-Aminobenzoic Acid | 0.05 |
| Ergocalciferol (D₂) | 0.1 |
| L-carnitine | 0.05 |
| DL-methionine-S-methyl- | 0.05 |
| sulfonium chloride (U) | |
| 2-Deoxyadenylic acid | 3.0 |
| (d-AMP) | |
| 5′-Thymidylic Acid (TMP) | 3.0 |
| 2′Deoxycitidine-5- | 3.0 |
| monophosphate (d-CMP) | |
| Carnosine | 25 |
| Citrulline | 50 |
| Sarcosine | 57 |
| CaCl₂ | 265 |
| Fe(NO₃)9H₂O | 0.72 |
| KCl | 400 |
| MgSO₄7 H₂O | 200 |
| NaCl | 5,850 |
| NaHCO₃ | 2,000 |
| NaH₂PO₄H₂O | 140 |
| Tricine | 900 |
| Hemin | 1 |
| HEPES | 2,000 |
| Glucose | 1,000 |
| D-ribose | 10 |
| 2-Deoxy-ribose | 10 |
| Cholecalciferol(D₃) | 0.1 |
| Biotin | 1 |
| Pyridoxamine | 0.05 |
| Pyridoxal | 1 |
| Cyanocobalamin(B₁₂) | 0.01 |
| Choline | 1 |
| Thiamine (B₁) | 1 |
| Inositol | 2 |
| α-Tocopherol | 0.01 |
| 3-phytylmenadione(K₁) | 0.01 |
| Menadione (K₃) | 0.01 |
| Retinol (A) | 0.14 |
| Riboflavin (B₂) | 0.1 |
| 6,8 Thiotic Acid | 0.01 |
| Pyridoxine (B₆) | 0.025 |
| Folic Acid | 1 |
| Niacinamide | 1 |
| Tetrahydrofolic Acid | 0.5 |
| Adenosine-5-Triphosphate (ATP) | 5.5 |
| 2′-Deoxyuridine-5-monophosphate | 3.0 |
| (d-UMP) | |
| 5′-Deoxyguanylic Acid (d-GMP) | 3.0 |
| Hydroxyproline | 262.5 |

The culture medium comprising the protozoan cells can then be treated in order to inactivate, and preferably kill, the cells. Upon isolation of those cells, the antigenic proteins can be purified therefrom and included in a pharmaceutically acceptable carrier, e.g., buffer solution, to create a second-generation immunotherapeutic agent. Preferably, the cells are inactivated or killed with a non-lysing agent, e.g., TLCK. The antigenic proteins of the present invention are particulate proteins that can be isolated from the cells using accepted methods. In a more specific embodiment the method of creating the second-generation immunotherapeutic agent of the present invention comprises the steps of (1) cultivating protozoa, preferably in the amastigote stage, in an appropriate culture medium; (2) treating said protozoan cells to inactivate or kill the cells; (3) isolating the treated cells; (4) extracting antigenic proteins from the isolated cells; and (5) formulating the second-generation immunotherapeutic agent composition by combining one or more isolated antigenic proteins with a pharmaceutically acceptable carrier, e.g., phosphate buffered saline (PBS). A preferred pharmaceutically acceptable carrier is a PBS solution having alumina present within the solution.

To cure psoriasis in patients with clinical and histopathological diagnosis of the disease, the first-generation polyvalent immunotherapeutic agent was administered intramuscularly, in the deltoid region, once a month, once every 15 days or once a week according to disease severity, for 7.6±6.0 months on average, at 500 μg/dose.

Furthermore to cure psoriasis a monovalent immunotherapeutic agent with each one of the Leishmania spp. present in the first-generation polyvalent immunotherapeutic agent was used as a subject composition with similar results to the polyvalent immunotherapeutic agent.

Furthermore to cure psoriasis a second-generation immunotherapeutic agent containing the protein fractions isolated by chromatographic means from the crude first-generation immunotherapeutic agent together with 0.1 ml alumina/mg protein was administered intramuscularly in the deltoid region once every 15 days for 3-4 doses at 200 μg/dose in 0.5 ml.

Following are examples which illustrate procedures for practicing the invention. These examples should not be construed as limiting. All percentages are by weight and all solvent mixture proportions are by volume unless otherwise noted.

### EXAMPLE 1

### Preparation of the Immunogen

Organisms of the genus leishmania are cultivated in the amastigote stage in the synthetic culture medium specified in Table 1, supplemented with 5% fetal bovine serum typically at about 30-34° C. (O'Daly et al., 1988, Acta Tropica (Basel), Vol. 45, pp. 109-126). For the second-generation immunotherapeutic agent, amastigotes at the stationary phase of growth were collected by centrifugation (800×g for 20 minutes at 4° C.), washed in Phosphate Buffered Saline (PBS), and incubated for 3 days at 30-34° C. in Eagle's MEM (Gibco) containing 150 μg of TLCK to inactivate the parasites as described (O'Daly et al., 1986, Acta Tropica (Basel), Vol. 43, pp. 225-236). After two washes with PBS (12.100×g for 10 minutes at 4° C.) 1×10⁸ parasites/ml were incubated in MEM containing 0.12% Nonidet-P-40 (NP40, Sigma) for 30 minutes at 4° C. to solubilize the surface antigens which were discarded (O'Daly et al., 1990 AM J Trop. Med. Hyg., Vol. 43, pp. 44-51). Particulate antigens were collected by centrifugation (12.100×g for 10 minutes at 4° C.), washed twice with PBS and sonicated for 5 minutes at 4° C. in a Sonifier Cell Disrupter (Model WI 85, Heath-Systems-Ultrasonic, Inc., Plainview, N.Y.) at the microtip limit of the output control at 50W. Protein content was determined by the method of Lowry (Lowry, 0. et al, 1951, J Biol. Chem., Vol. 193, pp. 265-275). The final monovalent first generation immunogen preparation contained 1 mg/ml of each Leishmania spp. antigens in PBS containing alumina (Aluminum hydroxide low viscosity gel REHYDRAGEL, Reheis Inc., New Jersey) at a concentration of 0.1 ml/mg (v/w) of parasite protein. Each step in the preparation of the immunogen was checked for sterility.

In another embodiment of the subject invention , particulate antigens were collected by centrifugation (12.100×g for 10 minutes at 4° C.), washed twice with PBS, dissolved in a solution containing 8 Molar Urea, 0.025 Tris (Tris-hydroxy-methyl-amino-methane) and sonicated for 5 minutes at 4° C. in a Sonifier Cell Disrupter (Model WI 85, Heath-Systems-Ultrasonic, Inc., Plainview, N.Y.) at the microtip limit of the output control at 50W. Protein fractions were separated by DEAE-chromatography.

The second-generation immunotherapeutic agent was prepared with each one of the seven protein fractions isolated after DEAE-chromatography of the subject composition containing only one leishmania specie as for example L.(V)brasiliensis or any other leishmania specie present in the crude first-generation immunotherapeutic agent. Protein content was determined by the method of Lowry (Lowry, 0. et al, 1951, J Biol. Chem., Vol. 193, pp. 265-275). Each protein fraction was dissolved in PBS and sonicated for 5 minutes at 4° C. in a Sonifier Cell Disrupter (Model WI 85, Heath-Systems-Ultrasonic, Inc., Plainview, N.Y.) at the microtip limit of the output control at 50W. Subsequently each fraction was filter-sterilized through 0.20 μm Millipore® filters. The final immunogen preparation contained 400 μg/ml of each of the antigenic fractions in PBS containing alumina (Aluminum hydroxide low viscosity gel REHYDRAGEL, Reheis Inc., New Jersey) at a concentration of 0.1 ml/mg (v/w) of the protein fraction. Each step in the preparation of the second generation immunogen was also checked for sterility.

Aliquots were incubated in ESM containing 5% Fetal Bovine Serum (FBS, Gibco) and in agar plates containing 12.5% (w/v) Bacto-Peptone (Difco), 12.5% (w/v) yeast extract (Becton Dickinson), 3.75% (w/v), glucose, and 3.75% (w/v) BBL agar (Becton Dickinson). Samples were incubated for 72 hours at 37° C. to detect fast growing bacteria and for 3 weeks at 26° C. for slow growing bacteria and fungus. Each batch of the immunogen was controlled by SDS-polyacrylamide gel electrophoresis to ensure consistency in the pattern of Leishmania protein bands. Each batch from the first and second generation immunotherapeutic agents was also tested with E-TOXATE (Sigma) for the presence of pyrogens. The first-generation immunogen was stable at 4° C. for at least 4 weeks.

### EXAMPLE 2

### Protein Components of the Immunogen

From the immunogen preparations obtained from the procedures described in Example 1 above, eight protein bands were identified via SDS-polyacrylamide gel electrophoresis of the TLCK-treated NP-40-extracted amastigotes from Leishmania(L)amazonensis, Leishmania(L)venezuelensis, Leishmania(V)brasiliensis, and Leishmania(L)chagasi, with apparent molecular weights of 21, 33, 44, 50, 55, 58, 65, and 77 kDa. In untreated entire amastigote extracts between 28 and 30 bands with molecular weights ranging from 29 to 96 kDa were observed in each Leishmania species, and major bands of 29, 34, 43, 58, and 65 kDa were observed.

The immunogen preparations of the second-generation immunotherapeutic agent, which contain protein fractions 3 and 4 obtained after DEAE-chromatography and total reduction and alkylation, had three bands with molecular weights of 73, 80, and 82 kDa.

### EXAMPLE 3

### Safety and Immunogenicity

The immunogenic composition comprising the proteins of the second-generation immunotherapeutic agent, described in Examples 1 and 2, above, was injected into a human volunteer at monthly intervals, beginning with 50 μg and increasing the dose by 50 μg each month, in order to determine the dose capable of inducing an IDR greater than 5 mm. This dose was found to be 200 μg. At both one month and six months after the last dose of immunotherapeutic agent, the following blood tests were performed on this volunteer: complete blood count; differential white blood cell count; urea; creatinin; sugar alkaline phosphatase; bilirubin; transaminases; cholesterol; triglycerides; C. reactive protein; serological tests such as VDRL, HIV, antinuclear antibodies, LE cells; and urine and fecal analysis. All the values were within normal limits, and no side effects were observed.

### EXAMPLE 4

### Preparation of Immunotherapeutic Agent Compositions

For the first-generation monovalent immunotherapeutic agent, cultivated amastigotes of each species of Leishmania were collected by centrifugation (800×g for 20 minutes at 4° C.), washed in Phosphate Buffered Saline (PBS) and incubated for 3 days at 30-34° C. in Eagles's MEM (Gibco) containing 150 μg of TLCK to inactivate the parasites as described, at 1×10⁸ parasites/ml. This step is preferably carried out when the amastigotes are in the stationary growth phase, after two washes with PBS (12.100×g for 10 minutes at 4° C.).

In a particularly preferred embodiment, preparation of a protective monovalent first generation immunogenic composition according to the subject invention comprises the following steps:

A) cultivating organisms of the genus Leishmania in the amastigote state in a synthetic culture medium containing the ingredients listed in Table 1 supplemented with 5% fetal bovine serum typically at about 30-34° C.;

B) subjecting organisms of the genus Leishmania in the amastigote stage, and at the stationary phase of growth, to a medium containing an amount of N-p-tosyl-L-Lysine chloromethyl ketone or a pharmacologically acceptable salt thereof effective to kill said cells;

C) isolating said killed cells;

D) extracting the surface proteins with the non-ionic detergent Nonidet p-40;

E) centrifugation of the preparation to isolate particulate antigens;

F) washing twice with PBS; and

G) forming an immunizing inoculum comprising said particulate antigens from said killed cells by resuspending them in phosphate buffered saline comprising alumina.

For the second generation immunotherapeutic agent composition, cultivated amastigotes were collected by centrifugation (800×g for 20 minutes at 4° C.), washed in Phosphate Buffered Saline (PBS) and incubated for 3 days at 30-34° C. in Eagles's MEM (Gibco) containing 150 μg of TLCK to inactivate the parasites as described, at 1×10⁸ parasites/ml. This step is preferably carried out when the amastigotes are in the stationary growth phase, after two washes with PBS (12.100×g for 10 minutes at 4° C.).

In a particularly preferred embodiment, preparation of a protective second generation immunogenic composition according to the subject invention comprises the following steps:

A) cultivating organisms of the genus Leishmania in the amastigote state in a synthetic culture medium containing the ingredients listed in Table 1 supplemented with 5% fetal bovine serum typically at about 30-34° C.;

B) subjecting organisms of the genus Leishmania in the amastigote stage and at the stationary phase of growth, to a medium containing an amount of N-p-tosyl-L-Lysine chloromethyl ketone or a pharmacologically acceptable salt thereof effective to kill said cells;

C) isolating said killed cells;

D) extracting the surface proteins with the non-ionic detergent Nonidet p-40;

E) centrifugation of the preparation to isolate particulate antigens;

F) washing twice with PBS,

G) dissolving in a solution containing 8 Molar Urea, 0.025 Molar Tris (Tris-hydroxy-methyl-amino-methane) and sonicating for 5 minutes at 4° C. in a Sonifier Cell Disrupter (Model WI 85, Heath-Systems-Ultrasonic, Inc., Plainview, N.Y.) at the microtip limit of the output control at 50W.

H) separating protein fractions in a DEAE-Sephadex column with a NaCl stepwise elution from 0.05-0.3 Molar NaCl concentration in a solution containing 8 Molar Urea, 0.025 Molar Tris pH 8.3; and

I) forming an immunizing inoculum comprising said particulate antigens from said killed cells by resuspending them in phosphate buffered saline comprising alumina.

In a particularly preferred embodiment, preparation of an immunogenic composition for clinical remission of psoriasis according to the second-generation subject invention comprises the following steps:

A) cultivating organisms of the genus Leishmania in the amastigote state in a synthetic culture medium containing the ingredients listed in Table 1 supplemented with 5% fetal bovine serum typically at about 34° C.;

B) subjecting organisms of the genus Leishmania in the amastigote stage and at the stationary phase of growth, to a medium containing an amount of N-p-tosyl-L-Lysine chloromethyl ketone or a pharmacologically acceptable salt thereof effective to kill said cells;

C) isolating said killed cells;

D) extracting the surface proteins with the non-ionic detergent Nonidet p-40;

E) DEAE Sephadex chromatography of particulate antigens from only one Leishmania specie, as for example L.(V)brasiliensis or any other Leishmania specie present in the first-generation immunotherapeutic agent;

F) isolating seven protein fractions in 8 Molar urea, 0.025 Molar Tris pH 8.3, separated using stepwise elution with 0.05-0.3 Molar NaCl;

G) dialysis vs distilled water and lyophylization of protein fractions;

H) dissolving the protein fractions in phosphate buffered saline;

I) determining protein content of the fractions by the method of Lowry (Lowry, 0. et al, 1951, J Biol. Chem., Vol. 193, pp. 265-275);

J) sonicating each protein fraction in phosphate buffered saline for 5 minutes at 4° C. in a Sonifier Cell Disrupter (Model WI 85, Heath-Systems-Ultrasonic, Inc., Plainview, N.Y.) at the microtip limit of the output control at 50W;

K) passing each fraction through 0.20 μm Millipore® filters; and

L) forming a second-generation immunizing inoculum comprising one or more of said protein fractions by resuspending the one or more fractions in phosphate buffered saline containing alumina.

### EXAMPLE 5

Treatment of Psoriasis With a First-Generation Polyvalent Immunotherapeutic Agent Containing L.(L)amazonensis, L.(L)venezuelensis, L.(L)brasiliensis, and L.(L) chagasi.

TABLE 2
Age groups in the study population.

| Age groups | Patients | % |
|---|---|---|
| [0-5] | 8 | 0.29 |
| [6-12] | 65 | 2.35 |
| [13-18] | 90 | 3.25 |
| [19-25] | 268 | 9.68 |
| [26-40] | 997 | 35.99 |
| [41-65] | 1196 | 43.18 |
| >65 | 146 | 5.27 |
| Total | 2770 | 100 |

The majority of patients (79.17%) were between 26-65 years of age with average age of 42.56±26.11 years and a range between 1 and 88 years of age.

TABLE 3
Characteristics of the study population.

| | | | | PATIENTS |
|---|---|---|---|---|
| | | | TIME | HAVING |
| | | | (YEARS) | RELATIVES |
| | | | WITH | WITH |
| | PATIENTS | AGE | PSORIASIS | PSORIASIS |
| Males | 1545 (55.8%) | 42.1 ± 14.3 | 11.2 ± 9.6 | 500 (32.3%) |
| Females | 1225 (44.2%) | 38.6 ± 15.3 | 12.0 ± 10.0 | 472 (38.5%) |
| Age ≦ 25 | 431 (15.6%) | 18.7 ± 5.5 | 6.1 ± 4.8 | 172 (39.9%) |
| Age ≧ 26 | 2339 (84.4%) | 44.6 ± 12.4 | 12.6 ± 10.2 | 800 (34.2%) |
| Total | 2770 | 40.6 ± 14.9 | 11.6 ± 9.8 | 972 (35.0%) |

35% had parents with psoriasis and the evolution time of the disease was 11.6±9.8 years, similar in males and females, with a range between 2 and 46 years.

TABLE 4
Clinical types of Psoriasis in the study population.

| | | | PLAQUE | | | | PLAQUE | |
|---|---|---|---|---|---|---|---|---|
| | | | + | PALM | ERYTHRO- | | + | |
| | PLAQUE | GUTATA | GUTATA | PLANTAR | DERMIA | INVERSE | ARTHRITIS | NAILS |
| Male | 1229 | 67 | 78 | 37 | 36 | 14 | 53 | 29 |
| | (56.1%) | (48.9%) | (56.9%) | (39.4%) | (72.0%) | (58.3%) | (55.2%) | (72.5%) |
| Female | 963 | 70 | 59 | 57 | 14 | 10 | 43 | 11 |
| | (43.9%) | (51.1%) | (43.1%) | (60.6%) | (28.0%) | (41.7%) | (44.8%) | (27.5%) |
| Age | 320 | 33 | 24 | 19 | 10 | 3 | 8 | 5 |
| ≦25 | (14.6%) | (24.1%) | (17.5%) | (20.2%) | (20%) | (12.5%) | (8.3%) | (12.5%) |
| Age | 1872 | 104 | 113 | 75 | 40 | 21 | 88 | 35 |
| ≧26 | (85.4%) | (75.9%) | (82.5%) | (79.8%) | (80%) | (87.5%) | (91.7%) | (87.5%) |
| Total | 2192 | 137 | 137 | 94 | 50 | 24 | 96 | 40 |
| | (79.1%) | (10.1%) | (10.1%) | (0.3%) | (1.8%) | (0.8%) | (3.4%) | (0.3%) |

92.6% had the clinical form of plaque psoriasis distributed in its pure form (79.1%) or associated with guttata (10.1%) or arthritis (3.4%); 10.1% had the Gutata pure form; 0.3% had the palmar and plantar form, 1.8% had Erythrodermia and 3.4% had psoriatic arthritis.

TABLE 5
Study population and response to vaccination in psoriatic
patients distributed by gender and age.

| | | PASI¹ | | | | | | |
|---|---|---|---|---|---|---|---|---|
| | | BEFORE | | | | | | |
| | | IMMUNOTHER- | REDUCTION OF PASI¹ AFTER | | | | | |
| | | APEUTIC | VACCINATION² | | | | | QUIT |
| | | AGENT | 100% | 99-70% | 69-40% | 39-10% | <10% | QUIT |
| Males | 1545 | 18.5 ± 16.9 | 323 | 600 | 185 | 105 | 55 | 272 |
| Females | 1225 | 13.7 ± 14.9 | (49.8%) | (57.0%) | (56.7%) | (61.8%) | (59.8%) | (56.5%) |
| | | | 325 | 453 | 141 | 65 | 37 | 209 |
| | | | (50.2%) | (43.0%) | (43.3%) | (38.2%) | (40.2%) | (43.5%) |
| Age ≦25 | 431 | 13.0 ± 14.7 | 131 | 150 | 50 | 24 | 12 | 69 |
| | | | (20.2%) | (14.2%) | (15.3%) | (14.1%) | (13.0%) | (14.3%) |
| Age ≧26 | 2339 | 17.0 ± 16.4 | 517 | 903 | 276 | 146 | 80 | 412 |
| | | | (79.8%) | (85.8%) | (84.7%) | (85.9%) | (87.0%) | (85.7%) |
| Total | 2770 | 16.4 ± 16.2 | 648 | 1053 | 326 | 170 | 92 | 481 |
| | | | (28.0%) | (46%) | (14.0%) | (7%) | (4%) | (17.4%) |

¹PASI = Psoriasis area and severity index
²Eight years of follow-up

Ninety six % of patients responded to treatment with a decrease in PASI values greater than 10%, and only 4% responded with a decrease in PASI values less than 10% from the initial PASI value before treatment. Twenty eight % had 100% remission of lesions, their disease disappeared completely, similar in males and females. Overall 74% had between 70-100% remission of lesions and 21% from 10-69% remission as compared with initial PASI values. 17.4% of volunteers quit treatment after 1-2 doses of immunotherapeutic agent (see below)

TABLE 6
Comparison of immunotherapeutic agent doses in each clinical remission group.
IMMUNOTHERAPEUTIC AGENT DOSES FOR
REDUCTION OF PASI AFTER VACCINATION¹

| | | 100% | 99-70% | 69-40% | 39-10% | <10% | QUIT |
|---|---|---|---|---|---|---|---|
| Males | 1545 | 7.7 ± 6.5 | 11.3 ± 10.8 | 9.2 ± 10.2 | 5.9 ± 4.5 | 6.1 ± 4.8 | 1.6 ± 1.1 |
| Females | 1225 | 7.5 ± 5.6 | 10.6 ± 10.0 | 8.8 ± 8.7 | 6.0 ± 4.6 | 5.9 ± 5.0 | 1.5 ± 1.1 |
| Age ≦ 25 | 431 | 6.5 ± 4.2 | 10.6 ± 10.0 | 8.2 ± 8.4 | 6.1 ± 6.1 | 6.5 ± 4.6 | 1.4 ± 0.6 |
| Age ≧ 26 | 2339 | 7.8 ± 6.4 | 11.1 ± 10.0 | 9.2 ± 9.8 | 5.9 ± 4.2 | 5.9 ± 5.0 | 1.7 ± 1.4 |
| Total | 2770 | 7.6 ± 6.0 | 11.0 ± 10.0 | 9.0 ± 9.6 | 6.0 ± 4.5 | 6.0 ± 4.9 | 1.7 ± 1.4 |

¹Subjects’ conditions were followed for eight years.

7.6±6.0 doses of immunotherapeutic agent were needed for 100% remission of psoriasis. The amount of doses in the groups with 70-90% and 40-69% remission were somewhat higher, reaching values of 11.0±10.0 and 9.0±9.6 respectively, which suggests that clinical remission depends mainly on the immunological response of the volunteer. The patient able to respond to the immunotherapeutic agent antigens is committed to do so since the beginning of treatment. The patient without response stays so, in spite of a higher number of immunotherapeutic agent doses.

TABLE 7
Appearance of relapses after clinical remission of Psoriasis.
APPEARANCE OF RELAPSES AFTER REMISSION IN 100% REMISSION GROUP

| | | | | | Time¹ | | | | % New |
|---|---|---|---|---|---|---|---|---|---|
| | | Doses for | Time¹ for | | from | PASI at | Doses for | Time¹ for | remissions |
| | Initial | 100% | 100% | PASI at | remission | new | new | new | after |
| Relapses | PASI | remission | remission | relapse | to relapse | remission | remission | remission | relapse |
| 188/648 | 21.0 ± 17.8 | 7.6 ± 6.0 | 7.0 ± 5.4 | 7.7 ± 10.1 | 15.4 ± 20.6 | 2.8 ± 3.3 | 7.1 ± 6.8 | 5.8 ± 4.9 | 161/188 |
| (28.9%) | | | | | | | | | (85.6%) |

¹months

From the 648 patients with total remission of lesions 188 (28.9%) volunteers had relapses of the disease after 15.4±20.6 months. PASI values at the time of relapse were ⅓ of the initial PASI value before treatment. The PASI at the new Clinical remission was considerable lower than the PASI at the time of relapse. The new remission occurred with 7.1±6.8 doses of immunotherapeutic agent after 5.8±4.9 weeks, a period of time lower than the time period observed in the first treatment cycle for Clinical remission of lesions. In this relapsing group 85.6% of patients had again remission of lesions after 6-7 doses of immunotherapeutic agent.

TABLE 8
Side effects after vaccination.

| SIGNS AT THE SITE OF INOCULATION | | | | SYSTEMIC | |
|---|---|---|---|---|---|
| Pain | Heat | Redness | Nodule | SYMPTOMS | NONE |
| 989(43.2%) | 484(21.1%) | 327(14.3%) | 535 (23.4%) | 588(25.7%) | 1233(53.9%) |

Minor side effects were observed at the site of inoculation in less than half of the patients with psoriasis, without difference due to gender or age. All of these disappeared within a few days. Results of the laboratory analysis of samples from 55 psoriasis patients who received 21.4±13.1 doses of first-generation immunotherapeutic agent are shown in Table 9. All values were found to be within normal ranges.

TABLE 9
Laboratory analysis in 55 psoriasis patients with 21.4 ± 13.1
doses of first-generation immunotherapeutic agent.

| | |
|---|---|
| White blood cell count/ul | 6003 ± 4165 |
| % Neutrophiles | 53.1 ± 13.3 |
| % Lymphocytes | 29.3 ± 13.3 |
| % Monocytes | 5.8 ± 3.8 |
| % Eosynophiles | 2.9 ± 2.3 |
| % Basophiles | 0.7 ± 0.6 |
| Red blood cell count × 10⁶/ul | 4.7 ± 0.6 |
| Hemoglobin g/dl | 13.3 ± 1.9 |
| Hematocrit (%) | 42.0 ± 5.9 |
| VCM(fl) | 91.6 ± 7.7 |
| MCH(pg) | 29.2 ± 3.2 |
| MCHC(g/dl) | 31.9 ± 1.0 |
| RDW-SD(fl) | 20.1 ± 14.9 |
| Platelets × 10⁶/ul | 250.3 ± 84.2 |
| UREA(mg/dl) | 19.7 ± 8.5 |
| CREATININE(mg/dl) | 0.9 ± 0.2 |
| URIC ACID(mg/dl) | 5.6 ± 1.6 |
| BLOOD SUGAR(mg/dl) | 89.8 ± 15.1 |
| TOTAL PROTEIN(g/dl) | 7.2 ± 0.8 |
| ALBUMINE(g/dl) | 3.8 ± 0.9 |
| GLOBULINES(g/dl) | 3.3 ± 0.8 |
| TRIGLICERIDES(mg/dl) | 161.0 ± 107.1 |
| LOW DENSITY LIPOPROTEINS (mg/dl) | 102.8 ± 44.5 |
| VERY LOW DENSITY | 35.0 ± 23.3 |
| LIPOPROTEINS(mg/dl) | |
| LACTIC ACID | 36.1 ± 13.2 |
| DEHYDROGENASE(mg/dl) | |
| PROTROMBIN TIME | 11.7 ± 1.3 |
| TROMBOPLASTIN PARTIAL TIME | 29.5 ± 6.5 |
| OXALOACETIC TRANSAMINASE(u/l) | 29.0 ± 14.1 |
| PYRUVIC TRANSAMINASE(u/l) | 26.1 ± 15.1 |
| SODIUM(mg/dl) | 144.9 ± 2.1 |
| POTASSIUM(mg/dl) | 4.2 ± 0.3 |
| CHLORINE(meq/l) | 105.3 ± 2.6 |
| CALCIUM(mg/dl) | 8.7 ± 0.3 |
| PHOSPHORUS(mg/dl) | 2.9 ± 0.4 |

### EXAMPLE 6

### Trial of First-Generation Monovalent Immunotherapeutic Agent

TABLE 10
Follow-up of a single blind trial after injection of psoriasis patients with one of
four Leishmania species present in the first-generation immunotherapeutic agent.

| | | IMMUNO- | | |
|---|---|---|---|---|
| | PASI | THERAPEUTIC | PASI | |
| | BEFORE | AGENT | AFTER | % PASI |
| LEISHMANIA SPECIE | TREATMENT | DOSES | TREATMENT | REDUCTION |
| L. (L) amazonensis | 6.4 | 3 | 1.4 | 78.1 |
| L. (L) amazonensis | 3.8 | 6 | 1.7 | 55.3 |
| L. (L) amazonensis | 3.6 | 3 | 1.4 | 61.1 |
| L. (L) amazonensis | 9.4 | 5 | 1.3 | 86.2 |
| L. (L) amazonensis | 2.3 | 3 | 0 | 100.0 |
| L. (V) brasiliensis | 36 | 2 | 15.4 | 57.2 |
| L. (V) brasiliensis | 11.9 | 2 | 1.8 | 84.9 |
| L. (V) brasiliensis | 13.9 | 5 | 6.4 | 54.0 |
| L. (V) brasiliensis | 5.8 | 4 | 1.9 | 67.2 |
| L. (L) chagasi | 2.8 | 5 | 0 | 100.0 |
| L. (L) chagasi | 52.2 | 3 | 0 | 100.0 |
| L. (L) chagasi | 10 | 3 | 4.5 | 55.0 |
| L. (L) venezuelensis | 15.6 | 3 | 5.3 | 66.0 |

Immunotherapeutic agents were also prepared using individual species of Leishmania from the first generation Immunotherapeutic agent and were subsequently tested for ability to induce Clinical remission of psoriasis lesions. The results in Table 15 clearly demonstrated that it is not necessary to prepare a mixture of four Leishmania species in the first generation Immunotherapeutic agent to obtain clinical remission of lesions in psoriasis patients. One Leishmania species is as effective as the mixture of four species used in the polyvalent immunotherapeutic agent to induce lower PASI values up to 100% after treatment. Thus, in every leishmania extract, there is a factor that inhibits the inflammation associated with psoriasis.

### EXAMPLE 7

### Formulation and Administration

The compounds of the invention are useful for various purposes, both therapeutic and non-therapeutic. Therapeutic application of the new compounds and compositions containing them can be contemplated to be accomplished by any suitable therapeutic method and technique presently or prospectively known to those skilled in the art. Further, the compounds of the invention have utility as starting materials or intermediates for the preparation of other useful compounds and compositions.

The dosage administered to a host in the above indications will be dependent upon the identity of the infection, the type of host involved, including the host's age, weight, and health, the existence and nature of concurrent treatments, if any, the frequency of treatment, and the therapeutic ratio.

The compounds of the subject invention can be formulated according to known methods for the preparation of pharmaceutical compositions. Formulations are described in detail in a number of sources which are well known and readily available to those skilled in the art. For example, Remington's Pharmaceutical Science by E. W. Martin describes formulations that can be used in connection with the subject invention. In general, the compositions of the subject invention will be formulated such that an effective amount of the bioactive compound(s) is (are) combined with a suitable carrier in order to facilitate effective administration of the composition.

### EXAMPLE 8

### Chromatographic Separation of Protein Fractions from Leishmania Species and Blastogenic Assay with Human Peripheral Blood Mononuclear Cells

Seven fractions were separated from the particulate Leishmania chagasi extract (PP75), the first component of the first-generation immunotherapeutic agent, after treatment of the respective amastigote parasites with TLCK and extraction with NP-40 as mentioned previously.

The fractions were tested in a blastogenic assay with peripheral blood mononuclear cells from psoriatic patients before and after vaccination according to methods routinely used in the art. For this example, 100 μl aliquots (triplicates) of each of the fractions dissolved in RPMI-1640 were pre-incubated in flat bottom microtiter plates (Falcon Plastics) with 2×10⁵ peripheral blood mononuclear cells, separated in HISTOPAQUE (Sigma) and resuspended in 100 μl of RPMI-1640 containing 20% heat inactivated fetal bovine serum under methods routine in the art. Concanavalin A was used as positive control of lymphocyte stimulation. 48 hours latter, 0.2 μCi/well of ³H-Thymidine was added in 10 μi aliquots and the samples were incubated for 18 additional hours. The cells were harvested on filter paper (Reeve Angel) using an automatic cell harvester (MASHII). The dried paper discs were placed in minivials with 2.5 ml Aquasol (NEN) and counted for 1 min. in a Packard Tri-Carb scintillation counter Model 3385. The stimulation index (S.I.) was calculated for each sample by dividing the experimental counts per minute (c.p.m.) by the control c.p.m. (cultures with fractions or mitogens/control cultures in culture medium alone). The results are illustrated in Tables 11-14 below.

TABLE 11
Peripheral blood mononuclear cells blastogenesis with fractions
from L(L). chagasi (PP75) before and after vaccination.

| | | | | CURED AFTER | |
|---|---|---|---|---|---|
| BEFORE VACCINATION | | | | VACCINATION | |
| | ug | n = 3 | | n = 5 | |
| DEAE | protein/ | cpm/well | S.I. | cpm/well | S.I. |
| Sephadex | well | X ± SD | X ± SD | X ± SD | X ± SD |
| Fraction 1 | 20 | 823 ± 215 | 1.90 ± 0.22 | 2044 ± 1825 | 3.22 ± 286 |
| No NaCl | 10 | 1297 ± 835 | 2.81 ± 1.5 | 1442 ± 1425 | 2.59 ± 276 |
| | 5 | 1587 ± 1429 | 3.40 ± 2.79 | 1424 ± 1150 | 2.44 ± 217 |
| | 2.5 | 627 ± 282 | 1.40 ± 0.41 | 1366 ± 951 | 2.27 ± 1.66 |
| Fraction 2 | 20 | 908 ± 103 | 2.22 ± 0.79 | 2643 ± 1798 | 4.36 ± 2.96 |
| 0.05 M Nacl | 10 | 821 ± 660 | 1.87 ± 1.1 | 1880 ± 1571 | 3.13 ± 2.83 |
| | 5 | 761 ± 324 | 1.73 ± 0.49 | 1627 ± 1137 | 2.75 ± 2.05 |
| | 2.5 | 532 ± 347 | 1.19 ± 0.63 | 1129 ± 900 | 1.94 ± 1.7 |
| Fraction 3 | 20 | 933 ± 728 | 2.03 ± 1.37 | 1735 ± 1764 | 3.03 ± 3.4 |
| 0.1 M NaCl | 10 | 941 ± 552 | 2.08 ± 1.77 | 1368 ± 1528 | 2.51 ± 2.94 |
| | 5 | 706 ± 376 | 1.57 ± 0.61 | 1360 ± 1681 | 2.45 ± 3.23 |
| | 2.5 | 717 ± 632 | 1.57 ± 1.21 | 1174 ± 1382 | 2.09 ± 2.66 |
| Fraction 4 | 20 | 674 ± 405 | 1.54 ± 0.74 | 2514 ± 1552 | 4.25 ± 2.73 |
| 0.15 M NaCl | 10 | 600 ± 305 | 1.38 ± 0.55 | 1541 ± 1548 | 2.74 ± 3.0 |
| | 5 | 767 ± 275 | 1.87 ± 0.84 | 1330 ± 1520 | 2.36 ± 2.93 |
| | 2.5 | 940 ± 346 | 2.35 ± 1.29 | 1216 ± 1225 | 2.16 ± 2.37 |
| Fraction 5 | 20 | 549 ± 197 | 1.24 ± 0.21 | 1411 ± 1629 | 2.52 ± 3.14 |
| 0.2 M NaCl | 10 | 472 ± 181 | 1.48 ± 0.58 | 1398 ± 1562 | 2.49 ± 3.01 |
| | 5 | 470 ± 205 | 1.06 ± 0.31 | 1095 ± 1023 | 1.94 ± 1.98 |
| | 2.5 | 353 ± 112 | 0.87 ± 0.03 | 1059 ± 907 | 1.86 ± 1.76 |
| Fraction 6 | 20 | 726 ± 126 | 1.70 ± 0.12 | 1448 ± 1127 | 2.52 ± 2.17 |
| 0.25 M NaCl | 10 | 558 ± 225 | 1.26 ± 0.31 | 1354 ± 818 | 2.46 ± 1.77 |
| | 5 | 778 ± 456 | 1.71 ± 0.78 | 1280 ± 752 | 2.28 ± 1.52 |
| | 2.5 | 688 ± 574 | 1.52 ± 1.09 | 927 ± 710 | 1.61 ± 1.36 |
| Fraction 7 | 20 | 694 ± 325 | 1.54 ± 0.48 | 1180 ± 747 | 1.91 ± 1.09 |
| 0.3 M NaCl | 10 | 676 ± 154 | 1.56 ± 0.10 | 1608 ± 1107 | 2.96 ± 2.27 |
| | 5 | 604 ± 217 | 1.39 ± 0.31 | 1325 ± 601 | 2.40 ± 1.32 |
| | 2.5 | 580 ± 315 | 1.28 ± 0.52 | 1466 ± 810 | 2.75 ± 1.89 |
| Concanavalin | 10 | 8452 ± 7470 | 23.12 ± 24.89 | 7988 ± 2805 | 13.58 ± 4.31 |
| A | 5 | 22479 ± 10642 | 55.05 ± 29.29 | 28011 ± 8183 | 52.67 ± 22.89 |
| Amastigote | 4 × 10⁶ | 795 ± 209 | 1.85 ± 0.32 | 2099 ± 1454 | |
| Parasites | 2 × 10⁶ | 741 ± 307 | 1.68 ± 0.45 | 1725 ± 1028 | 3.40 ± 2.02 |
| | | | | | 2.75 ± 0.99 |
| Culture | | 323 ± 79 | 1.0 ± 0.2 | 987 ± 226 | 1.0 ± 0.3 |
| medium | | | | | |

The group of patients before vaccination had S.I.>1.0. These values increased markedly after vaccination. Results of the statistical analysis of both groups are as follows:

| Parameter | Before vaccination | After vaccination |
|---|---|---|
| Mean | 1.697143 | 2.571072 |
| # points | 28 | 28 |
| Std deviation | .5298834 | .6259645 |
| Std error | .1001386 | .1182962 |
| Minimum | .87 | 1.61 |
| Maximum | 3.4 | 4.36 |

Paired t test:
Mean difference = −.8739286 (Mean of paired differences)
95% confidence interval of the difference: −1.150029 to −0.5978283
Two-tailed p value is <0.0001 --- extremely significant-

These results demonstrate that, after vaccination of psoriatic patients with any of the fractions of the L.(L)chagasi extract, lymphocytes are significantly stimulated. Higher stimulation index was observed with fractions 3 and 4 as well as live amastigotes.

Seven fractions were separated from the particulate L(V) brasiliensis extract (PMH27), a second component of the first-generation immunotherapeutic agent, after treatment of the respective amastigote parasites with TLCK and extraction with NP-40 as mentioned previously.

TABLE 12
Peripheral blood mononuclear cells blastogenesis with fractions
from L. (V)brasiliensis (PMH27) before and after vaccination.

| | | BEFORE | | BEFORE | | AFTER | |
|---|---|---|---|---|---|---|---|
| | | VACCINATION | | VACCINATION | | VACCINATION | |
| | ug | N = 3, S.I. < 1.0 | | N = 2, S.I. > 1.0 | | CURED, N = 3 | |
| DEAE | protein/ | cpm/well | S.I. | cpm/well | S.I. | cpm/well | S.I. |
| Sephadex | well | X ± SD | X ± SD | X ± SD | X ± SD | X ± SD | X ± SD |
| Fraction 1 | 20.00 | 379 ± 23 | 0.85 ± 0.35 | 812 ± 416 | 1.74 ± 0.47 | 1074 ± 509 | 1.98 ± 0.86 |
| No NaCl | 10.00 | 391 ± 65 | 0.84 ± 0.17 | 1423 ± 1173 | 2.99 ± 1.78 | 1945 ± 2481 | 3.51 ± 4.41 |
| | 5.00 | 491 ± 115 | 1.10 ± 0.46 | 1391 ± 1120 | 3.04 ± 1.8 | 683 ± 224 | 1.26 ± 0.36 |
| | 2.50 | 376 ± 105 | 0.80 ± 0.18 | 879 ± 137 | 2.06 ± 0.59 | 650 ± 240 | 1.19 ± 0.39 |
| Fraction 2 | 20.00 | 902 ± 775 | 1.76 ± 1.28 | 2686 ± 2098 | 5.88 ± 3.4 | 2157 ± 267 | 4.01 ± 0.48 |
| 0.05 M Nacl | 10.00 | 709 ± 555 | 1.39 ± 0.89 | 1971 ± 399 | 5.05 ± 3.13 | 1428 ± 351 | 2.65 ± 0.61 |
| | 5.00 | 1385 ± 639 | 3.12 ± 1.65 | 1690 ± 203 | 4.30 ± 2.51 | 1911 ± 533 | 3.56 ± 1.01 |
| | 2.50 | 1117 ± 1004 | 2.19 ± 1.67 | 2887 ± 716 | 6.59 ± 1.28 | 1661 ± 1225 | 3.01 ± 2.15 |
| Fraction 3 | 20.00 | 263 ± 21 | 0.58 ± 0.19 | 1028 ± 163 | 2.59 ± 1.46 | 2237 ± 1002 | 4.13 ± 1.75 |
| 0.1 M NaCl | 10.00 | 231 ± 65 | 0.48 ± 0.07 | 928 ± 314 | 2.06 ± 0.25 | 1633 ± 594 | 3.01 ± 1.0 |
| | 5.00 | 207 ± 44 | 0.44 ± 0.05 | 787 ± 365 | 1.74 ± 0.47 | 1479 ± 983 | 2.74 ± 1.76 |
| | 2.50 | 200 ± 41 | 0.42 ± 0.04 | 618 ± 252 | 1.40 ± 0.41 | 1140 ± 767 | 2.09 ± 1.36 |
| Fraction 4 | 20.00 | 251 ± 51 | 0.58 ± 0.30 | 1046 ± 335 | 2.41 ± 0.7 | 946 ± 513 | 2.75 ± 0.92 |
| 0.15 M NaCl | 10.00 | 260 ± 87 | 0.54 ± 0.09 | 1272 ± 767 | 2.74 ± 1.04 | 1118 ± 349 | 2.06 ± 0.56 |
| | 5.00 | 279 ± 67 | 0.59 ± 0.08 | 1442 ± 821 | 3.27 ± 1.42 | 915 ± 362 | 1.68 ± 0.6 |
| | 2.50 | 233 ± 37 | 0.50 ± 0.13 | 1335 ± 783 | 2.83 ± 0.96 | 930 ± 414 | 1.71 ± 0.71 |
| Fraction 5 | 20.00 | 232 ± 59 | 0.49 ± 0.05 | 669 ± 157 | 1.54 ± 0.39 | 1306 ± 365 | 2.42 ± 0.62 |
| 0.2 M NaCl | 10.00 | 275 ± 37 | 0.62 ± 0.25 | 577 ± 170 | 1.29 ± 0.12 | 911 ± 196 | 1.69 ± 0.33 |
| | 5.00 | 252 ± 64 | 0.54 ± 0.11 | 660 ± 228 | 1.45 ± 0.1 | 753 ± 240 | 1.38 ± 0.38 |
| | 2.50 | 285 ± 135 | 0.58 ± 0.16 | 704 ± 94 | 1.69 ± 0.65 | 822 ± 323 | 1.51 ± 0.53 |
| Fraction 6 | 20.00 | 233 ± 84 | 0.48 ± 0.10 | 873 ± 566 | 1.81 ± 0.76 | 909 ± 123 | 1.68 ± 0.17 |
| 0.25 M NaCl | 10.00 | 372 ± 215 | 0.74 ± 0.3 | 895 ± 705 | 1.89 ± 1.08 | 1043 ± 406 | 1.97 ± 0.88 |
| | 5.00 | 436 ± 258 | 0.87 ± 0.37 | 1053 ± 427 | 2.54 ± 1.24 | 971 ± 201 | 1.82 ± 0.48 |
| | 2.50 | 310 ± 76 | 0.66 ± 0.14 | 1308 ± 489 | 3.24 ± 1.82 | 773 ± 206 | 1.43 ± 0.32 |
| Fraction 7 | 20.00 | 1004 ± 881 | 2.03 ± 1.42 | 1406 ± 277 | 3.26 ± 0.8 | 1413 ± 638 | 2.60 ± 1.08 |
| 0.3 M NaCl | 10.00 | 2114 ± 1366 | 4.14 ± 1.92 | 2545 ± 1170 | 5.52 ± 1.16 | 1955 ± 472 | 3.62 ± 0.75 |
| | 5.00 | 2295 ± 2915 | 4.19 ± 1.03 | 2549 ± 1291 | 5.71 ± 2.02 | 931 ± 179 | 1.74 ± 0.41 |
| | 2.50 | 349 ± 206 | 0.70 ± 0.28 | 1479 ± 1503 | 2.99 ± 2.42 | 558 ± 186 | 1.02 ± 0.3 |
| Concanavalin | 10.00 | 17443 ± 9651 | 41.98 ± 32.89 | 7180 ± 2557 | 19.31 ± 15.19 | 20051 ± 12578 | 37.29 ± 22.55 |
| A | 5.00 | 30323 ± 2242 | 67.32 ± 21.79 | 14665 ± 12253 | 31.21 ± 19.01 | 33798 ± 4946 | 62.89 ± 8.16 |
| Amastigote | 4 × 10⁶ | 1035 ± 526 | 2.19 ± 0.87 | 2327 ± 974 | 5.17 ± 1.23 | 5128 ± 826 | 9.52 ± 1.21 |
| parasites | 2 × 10⁶ | 395 ± 147 | 1 ± 0.05 | 2427 ± 1968 | 4.37 ± 3.52 | 520 ± 33 | 0.90 ± 0.5 |
| Culture | | 390 ± 114 | 1.0 ± 0 | 557 ± 49 | 1.0 ± 0.3 | 580 ± 0 | 1.0 ± 0 |
| medium | | | | | | | |

In Table 12, two groups of patients were evident before vaccination, specifically, one group with S.I.<1.0 and another group with S.I.>1.0. The group of patients cured after vaccination had markedly increased values when compared with either of these groups before vaccination. Results of the statistical analysis are as follows:

Group with S.I. < 1.0

| Parameter | Before vaccination | After vaccination |
|---|---|---|
| Mean | 1.150714 | 2.257857 |
| # points | 28 | 28 |
| Std deviation | 1.062052 | .8876538 |
| Std error | .200709 | .1677508 |
| Minimum | .42 | 1.02 |
| Maximum | 4.19 | 4.13 |

Paired t test:
Mean difference = -1.107143(Mean of paired differences)
95% confidence interval of the difference: −1.534381 to −.6799043
Two-tailed p value is <0.0001 --- extremely significant-

Group with S.I > 1.0

| Parameter | Before vaccination | After vaccination |
|---|---|---|
| Mean | 2.986429 | 2.257857 |
| # points | 28 | 28 |
| Std deviation | 1.504479 | .8876538 |
| Std error | .2843199 | .1677508 |
| Minimum | 1.29 | 1.02 |
| Maximum | 6.59 | 4.13 |

Unpaired t test:
Mean difference = −.7285719 (Mean of B minus mean of A)
95% confidence interval of the difference: −1.3904 to −6.674413E−02
Two-tailed p value is <0.0316 --- significant-

These results demonstrate that lymphocytes from both of the pre-vaccination groups are significantly stimulated by vaccination with any of the fractions of the L.(V)brasiliensisextract. Higher stimulation index was observed with fractions 3 and 4 as well as live amastigotes.

Six fractions were separated from the particulate L.(L)venezuelensis extract (PMH16), the third component of the first-generation immunotherapeutic agent, after treatment of the respective amastigote parasites with TLCK and extraction with NP-40 as mentioned previously.

TABLE 13
Peripheral blood mononuclear cells blastogenesis with fractions
from L. (L) venezuelensis (PMH16) before and after vaccination.

| | | BEFORE | | BEFORE | | CURED AFTER | |
|---|---|---|---|---|---|---|---|
| | | VACCINATION | | VACCINATION | | VACCINATION | |
| | ug | n = 5, S.I. < 1.0 | | n = 2, S.I. > 1.0 | | n = 2 | |
| DEAE | protein/ | cpm/well | S.I. | cpm/well | S.I. | cpm/well | S.I. |
| Sephadex | well | X ± SD | X ± SD | X ± SD | X ± SD | X ± SD | X ± SD |
| Fraction 1 | 20.00 | 1617 ± 1622 | 1.95 ± 1.51 | 480 ± 92 | 0.89 ± 0.3 | 826 ± 104 | 1.78 ± 0.42 |
| No NaCl | 10.00 | 1455 ± 1241 | 1.82 ± 1.03 | 737 ± 57 | 1.36 ± 0.72 | 518 ± 74 | 1.11 ± 0.62 |
| | 5.00 | 1222 ± 905 | 1.57 ± 0.66 | 488 ± 75 | 0.90 ± 0.43 | 551 ± 42 | 1.1 ± 0.63 |
| | 2.50 | 1376 ± 1147 | 1.73 ± 0.93 | 468 ± 63 | 0.87 ± 0.27 | 377 ± 27 | 0.812 ± 0.3 |
| Fraction 2 | 20.00 | 1579 ± 1259 | 1.77 ± 1.39 | 1997 ± 1965 | 1.86 ± 1.05 | 2201 ± 419 | 3.52 ± 0.82 |
| 0.05 M Nacl | 10.00 | 1371 ± 476 | 1.65 ± 0.93 | 2163 ± 489 | 2.65 ± 102 | 1840 ± 1895 | 2.41 ± 1.89 |
| | 5.00 | 1003 ± 455 | 1.11 ± 0.48 | 1521 ± 1235 | 1.52 ± 0.46 | 1238 ± 1093 | 1.68 ± 0.97 |
| | 2.50 | 785 ± 164 | 0.87 ± 0.19 | 1398 ± 1309 | 1.33 ± 0.65 | 1259 ± 1256 | 1.66 ± 1.23 |
| Fraction 3 | 20.00 | 896 ± 358 | 0.98 ± 0.36 | 1859 ± 2160 | 1.61 ± 1.41 | 3681 ± 170 | 6.08 ± 2.25 |
| 0.1 M NaCl | 10.00 | 948 ± 594 | 1.02 ± 0.53 | 4858 ± 6397 | 3.92 ± 4.67 | 4178 ± 1306 | 7.41 ± 5.06 |
| | 5.00 | 689 ± 268 | 0.77 ± 0.35 | 1299 ± 1182 | 1.25 ± 0.56 | 3802 ± 1792 | 6.96 ± 5.61 |
| | 2.50 | 707 ± 302 | 0.77 ± 0.29 | 1760 ± 1967 | 1.55 ± 1.23 | 2775 ± 276 | 4.53 ± 1.45 |
| Fraction 4 | 20.00 | 848 ± 401 | 0.89 ± 0.25 | 1859 ± 1316 | 1.93 ± 0.3 | 2797 ± 1204 | 4.24 ± 0.08 |
| 0.15 M NaCl | 10.00 | 886 ± 810 | 0.91 ± 0.58 | 1930 ± 95 | 2.49 ± 1.35 | 3734 ± 2376 | 5.40 ± 1.39 |
| | 5.00 | 1105 ± 1103 | 1.07 ± 0.76 | 2024 ± 402 | 2.81 ± 2.08 | 1539 ± 182 | 2.63 ± 1.37 |
| | 2.50 | 826 ± 479 | 0.90 ± 0.49 | 1065 ± 794 | 1.09 ± 0.23 | 1151 ± 442 | 1.76 ± 0.06 |
| Fraction 5 | 20.00 | 1087 ± 618 | 0.91 ± 0.53 | 2416 ± 651 | 2.92 ± 1.0 | 2612 ± 1583 | 4.90 ± 4.44 |
| 0.2 M NaCl | 10.00 | 848 ± 601 | 1.14 ± 1.26 | 1912 ± 427 | 2.34 ± 0.91 | 1648 ± 165 | 2.80 ± 1.41 |
| | 5.00 | 587 ± 230 | 0.65 ± 0.22 | 2092 ± 108 | 2.78 ± 1.75 | 2324 ± 2119 | 4.60 ± 5.13 |
| | 2.50 | 553 ± 186 | 0.62 ± 0.21 | 1434 ± 842 | 1.56 ± 0.1 | 1235 ± 150 | 2.11 ± 1.1 |
| Fraction 6 | 20.00 | 767 ± 15 | 1.14 ± 0.42 | 129 ± 15 | 2.40 ± 0.57 | 1583 ± 640 | 3.41 ± 1.5 |
| 0.25 M NaCl | 10.00 | 515 ± 91 | 0.74 ± 0.16 | 852 ± 22 | 1.58 ± 0.63 | 1659 ± 315 | 3.57 ± 0.95 |
| | 5.00 | 374 ± 31 | 0.55 ± 0.17 | 577 ± 46 | 1.07 ± 0.38 | 592 ± 92 | 1.27 ± 0.47 |
| | 2.50 | 422 ± 17 | 0.62 ± 0.21 | 446 ± 24 | 0.82 ± 0.59 | 491 ± 27 | 1.05 ± 0.35 |
| Concanavalin | 20.00 | 29329 ± 13560 | 134 ± 237 | 22781 ± 8014 | 23.01 ± 6.19 | 10028 ± 4113 | 21.61 ± 11.25 |
| A | 10.00 | 34463 ± 10198 | 40 ± 17 | 48480 ± 8611 | 66.96 ± 48 | 24309 ± 12540 | 52.39 ± 36 |
| | 5.00 | 33799 ± 7901 | 52 ± 31 | 49409 ± 7469 | 63.8 ± 39 | 43290 ± 6532 | 93.29 ± 22.5 |
| | 2.50 | 35113 ± 1040 | 52.28 ± 18 | 42183 ± 10112 | 58.2 ± 19 | 35165 ± 4526 | 75.78 ± 36.5 |
| Amastigote | 4 × 10⁶ | 1315 ± 404 | 1.55 ± 0.78 | 2933 ± 429 | 3.22 ± 0.11 | 2500 ± 715 | 5.38 ± 1.2 |
| parasites | 2 × 10⁶ | 1665 ± 452 | 2.36 ± 0.27 | | | 3032 ± 1256 | 6.5 ± 3.4 |
| Culture | | 914 ± 237 | 1.0 ± 0.3 | 539 ± 74 | 1.0 ± 0.2 | 464 ± 59 | 1.0 ± 0 |
| medium | | | | | | | |

In Table 13 two groups of patients are evident before vaccination, specifically, one group with S.I.<1.0 and another group with S.I.>1.0. The group of patients cured after vaccination had markedly increased values when compared with either of these pre-vaccination groups.

Results of the statistical analyses are as follows.

Group with S.I. < 1.0

| Parameter | Before vaccination | After vaccination |
|---|---|---|
| Mean | 1.089583 | 3.205 |
| # points | 24 | 24 |
| Std deviation | .4250269 | 1.938181 |
| Std error | 8.675825 E−02. | .3956296 |
| Minimum | .55 | .81 |
| Maximum | 1.95 | 7.41 |

Paired t test:
Mean difference = −2.115417 (Mean of paired differences)
95% confidence interval of the difference: −3.008944 to −1.22189
Two-tailed p value is <0.0001 --- extremely significant-

Group with S.I. > 1.0

| Parameter | Before vaccination | After vaccination |
|---|---|---|
| Mean | 1.814167 | 3.205 |
| # points | 24 | 24 |
| Std deviation | .8092286 | 1.938181 |
| Std error | .165183 | .3956296 |
| Minimum | .83 | .81 |
| Maximum | 3.92 | 7.41 |

Unpaired t test:
Mean difference = −.7285719 (Mean of B minus mean of A)
95% confidence interval of the difference: −1.3904 to −6.674413E−02
Two-tailed p value is <0.0316 --- significant-

These results demonstrate that lymphocytes from both pre-vaccination groups of patients are significantly stimulated by vaccination with any of the fractions of the L.(L)venezuelensis extract. Higher stimulation index was observed with fractions 3 and 4 as well as live amastigotes.

Seven fractions were separated from the L.(L)amazonensis extract (PMH8), the fourth component of the first-generation immunotherapeutic agent, after treatment of the respective amastigote parasites with TLCK and extraction with NP-40 as mentioned previously.

TABLE 14
Peripheral blood mononuclear cells blastogenesis with fractions
from L.(L)amazonensis (PMH8), before and after vaccination.

| | | BEFORE | | BEFORE | | CURED AFTER | |
|---|---|---|---|---|---|---|---|
| | | VACCINATION | | VACCINATION | | VACCINATION | |
| | ug | n = 4, S.I. < 1.0 | | n = 4, S.I. > 1.0 | | n = 4 | |
| DEAE | protein/ | cpm/well | S.I. | cpm/well | S.I. | cpm/well | S.I. |
| Sephadex | well | X ± SD | X ± SD | X ± SD | X ± SD | X ± SD | X ± SD |
| Fraction 1 | 20.00 | 450 ± 22 | 0.84 ± 0.1 | 265 ± 22 | 1 ± 0 | 1525 ± 1374 | 1.48 ± 0.97 |
| No NaCl | 10.00 | 371 ± 19 | 0.70 ± 0.35 | 285 ± 45 | 1.07 ± 0.3 | 1392 ± 1222 | 1.95 ± 1.27 |
| | 5.00 | 392 ± 45 | 0.74 ± 0.14 | 448 ± 17 | 1.69 ± 0.45 | 1211 ± 584 | 1.79 ± 0.46 |
| | 2.50 | 480 ± 62 | 0.9 ± 0.32 | 311 ± 42 | 1.17 ± 0.25 | 1152 ± 733 | 1.67 ± 0.71 |
| Fraction 2 | 20.00 | 735 ± 405 | 0.64 ± 0.16 | 3576 ± 4474 | 3.37 ± 2.57 | 1614 ± 1540 | 2.22 ± 1.66 |
| 0.05 M Nacl | 10.00 | 574 ± 356 | 0.59 ± 0.26 | 1107 ± 1066 | 1.38 ± 0.07 | 1939 ± 1297 | 2.24 ± 1.35 |
| | 5.00 | 580 ± 238 | 0.60 ± 0.13 | 1181 ± 1311 | 1.29 ± 0.47 | 1569 ± 970 | 2.28 ± 1.10 |
| | 2.50 | 522 ± 68 | 0.61 ± 0.25 | 1173 ± 1217 | 1.37 ± 0.27 | 1180 ± 1215 | 1.61 ± 1.3 |
| Fraction 3 | 20.00 | 885 ± 928 | 0.84 ± 0.61 | 1488 ± 1524 | 1.76 ± 0.3 | 1716 ± 1355 | 2.49 ± 1.49 |
| 0.1 M NaCl | 10.00 | 585 ± 164 | 0.59 ± 0.16 | 1582 ± 285 | 3.29 ± 2.71 | 2453 ± 2095 | 3.56 ± 2.31 |
| | 5.00 | 676 ± 284 | 0.75 ± 0.08 | 1073 ± 850 | 1.53 ± 0.35 | 807 ± 423 | 1.21 ± 0.42 |
| | 2.50 | 593 ± 398 | 0.81 ± 0.51 | 1267 ± 1003 | 1.81 ± 0.41 | 807 ± 452 | 1.20 ± 0.45 |
| Fraction 4 | 20.00 | 733 ± 64 | 1.38 ± 0.6 | 349 ± 15 | 1.31 ± 0.4 | 1759 ± 374 | 2.80 ± 0.74 |
| 0.15 M NaCl | 10.00 | 428 ± 26 | 0.84 ± 0.2 | 1293 ± 254 | 4.87 ± 0.52 | 1424 ± 152 | 1.57 ± 0.72 |
| | 5.00 | 297 ± 37 | 0.56 ± 0.15 | 627 ± 90 | 2.36 ± 0.45 | 927 ± 97 | 1.49 ± 0.4 |
| | 2.50 | 374 ± 29 | 0.70 ± 0.14 | 397 ± 26 | 1.49 ± 0.65 | 939 ± 559 | 1.41 ± 0.78 |
| Fraction 5 | 20.00 | 236 ± 16 | 0.44 ± 0.2 | 287 ± 46 | 1.08 ± 0.4 | 442 ± 226 | 0.74 ± 0.5 |
| 0.2 M NaCl | 10.00 | 383 ± 45 | 0.72 ± 0.15 | 231 ± 26 | 0.87 ± 0.22 | 421 ± 127 | 0.67 ± 0.24 |
| | 5.00 | 250 ± 39 | 0.47 ± 0.18 | 236 ± 39 | 0.89 ± 0.16 | 280 ± 55 | 0.44 ± 0.09 |
| | 2.50 | 276 ± 52 | 0.52 ± 0.27 | 302 ± 11 | 1.13 ± 0.45 | 334 ± 43 | 0.54 ± 0.17 |
| Fraction 6 | 20.00 | 251 ± 45 | 0.47 ± 0.14 | 265 ± 93 | 1 ± 0 | 779 ± 354 | 1.05 ± 0.11 |
| 0.25 M NaCl | 10.00 | 284 ± 17 | 0.53 ± 0.21 | 250 ± 42 | 0.94 ± 0.4 | 679 ± 235 | 1.03 ± 0.24 |
| | 5.00 | 262 ± 26 | 0.49 ± 0.11 | 323 ± 196 | 1.22 ± 0.38 | 532 ± 222 | 1.01 ± 0.26 |
| | 2.50 | 264 ± 32 | 0.49 ± 0.12 | 298 ± 29 | 1.12 ± 0.6 | 450 ± 236 | 0.73 ± 0.48 |
| Fraction 7 | 20.00 | 1038 ± 453 | 2.03 ± 0.5 | 522 ± 125 | 1.97 ± 0.5 | 1074 ± 658 | 1.62 ± 0.92 |
| 0.3 M NaCl | 10.00 | 507 ± 144 | 0.96 ± 0.32 | 697 ± 74 | 2.63 ± 0.58 | 668 ± 275 | 1.01 ± 0.27 |
| | 5.00 | 395 ± 61 | 0.74 ± 0.37 | 611 ± 85 | 2.30 ± 0.45 | 898 ± 674 | 1.37 ± 0.9 |
| | 2.50 | 485 ± 56 | 0.91 ± 0.26 | 626 ± 92 | 2.36 ± 0.62 | 732 ± 403 | 1.09 ± 0.52 |
| Concanavalin | 10 | 33179 ± 9137 | 37.67 ± 16.2 | 25676 ± 13921 | 43.56 ± 22.88 | 18975 ± 10149 | 28.27 ± 11.54 |
| A | 5.00 | 31012 ± 12118 | 36.31 ± 7.42 | 39742 ± 3747 | 86.32 ± 75.86 | 17425 ± 7521 | 26.31 ± 8.18 |
| Amastigote | 4 × 10⁶ | 1775 ± 702 | 2.15 ± 0.67 | 2271 ± 2564 | 2.44 ± 1.0 | 3027 ± 2268 | 4.33 ± 2.69 |
| Parasites | | | | | | | |
| Culture | | 510 ± 89 | 1.00 ± 0.1 | 265 ± 59 | 1.0 ± 0 | 529 ± 67 | 1.0 ± 0 |
| medium | | | | | | | |

In Table 14, two groups of patients are evident before vaccination, specifically, one group with S.I.<1.0 and another group with S.I.>1.0. The group of patients cured after vaccination had markedly increased values when compared with either of these pre-vaccination groups.

Results of the statistical analysis are as follows:

Group with S.I. < 1.0

| Parameter | Before vaccination | After vaccination |
|---|---|---|
| Mean | .7007408 | 1.271786 |
| # points | 27 | 28 |
| Std deviation | .2043736 | .5430509 |
| Std error | .0393317. | .102627 |
| Minimum | .45 | .47 |
| Maximum | 1.39 | 3.15 |

Unpaired t test:
Mean difference = −.5710449 (Mean of paired differences)
95% confidence interval of the difference: .3475174 to .7945725
Two-tailed p value is <0.0001 --- extremely significant-

Group with S.I > 1.0

| Parameter | Before vaccination | After vaccination |
|---|---|---|
| Mean | 1.726786 | 1.271786 |
| # points | 28 | 28 |
| Std deviation | .9234719 | .5430509 |
| Std error | .1745198 | .102627 |
| Minimum | .88 | .47 |
| Maximum | 4.88 | 3.15 |

Unpaired t test:
Mean difference = −.4549999 (Mean of B minus mean of A)
95% confidence interval of the difference: −.8608927 to −4.910712E−02
Two-tailed p value is <0.0287 --- significant-

These results demonstrate that lymphocytes from both pre-vaccination groups of patients are significantly stimulated by vaccination with any of the fractions of the L.(L)amazonensis extract. Higher stimulation index was observed with fractions 3 and 4 as well as live amastigotes. In summary, each of the blastogenesis experiments demonstrate that vaccination with any of the protein fractions from each of the leishmania species included in the first-generation immunotherapeutic agent, and particularly fractions 3 and 4, results in significant stimulation of lymphocytes. The stimulated lymphocytes produce cytokines that can inhibit the inflammatory response in psoriatic patients, thus inducing clinical remission of the psoriatic lesions.

### EXAMPLE 14

### Humoral Immunity in Psoriatic Patients

TABLE 15
ELISA in psoriatic patients before and after vaccination.
(O'Daly et al. 1994 Acta Tropica 56: 265-287)

| | Immunother- | | | | |
|---|---|---|---|---|---|
| Number of | apeutic agent | Optical Density 405 nm (Average ± S.D.) | | | |
| Patients | Doses | La | Lv | Lb | Lch |
| 36 | 0 | 0.21 ± 0.20 | 0.40 ± 0.18 | 0.37 ± 0.22 | 0.35 ± 0.18 |
| 13 | 1 | 0.12 ± 0.00 | 0.21 ± 0.09 | 0.22 ± 0.10 | 0.19 ± 0.07 |
| 18 | 2 | 0.37 ± 0.27 | 0.35 ± 0.16 | 0.32 ± 0.17 | 0.33 ± 0.14 |
| 17 | 3 | 0.47 ± 0.22 | 0.38 ± 0.15 | 0.41 ± 0.20 | 0.36 ± 0.10 |
| 12 | 4 | 0.41 ± 0.28 | 0.30 ± 0.11 | 0.22 ± 0.09 | 0.26 ± 0.03 |
| 12 | 6 | 0.38 ± 0.27 | 0.34 ± 0.18 | 0.36 ± 0.05 | 0.30 ± 0.01 |
| 16 | Active | 0.91 ± 0.27 | 0.82 ± 0.21 | 0.77 ± 0.24 | 0.92 ± 0.26 |
| | leishmaniasis | | | | |

La: Leishmania amazonensis;
Lv: L. venezuelensis
Lb: L. brasiliensis;
Lch: L. chagasi

Sera from psoriasis patients were assayed before and after vaccination with an Enzyme Linked Immunosorbent Assay (ELISA), the results of which are shown in Table 15. No difference in optical density values was observed between pre-vaccination and post-vaccination samples up to clinical remission of lesions after six doses of the first-generation immunotherapeutic agent. The cut-off point for a positive reaction was 0.5 units. The only positive sera belonged to samples from patients with active leishmaniasis. This demonstrates that the first-generation immunotherapeutic agent is not inducing Humoral Immunity or TH2 responses.

### EXAMPLE 15

### Cellular Immunity in Psoriatic Patients

TABLE 16
Intradermic reaction to antigenic fractions in patients after clinical remission of psoriasis.
IDR DIAMETER (mm)

| | | CHROMATOGRAPHY FRACTIONS | | | | | | | |
|---|---|---|---|---|---|---|---|---|---|
| Parasite | Patients | 1 | 2 | 3 | 4 | 5 | 6 | 7 | P¹ |
| L. (L)chagasi | 15 | 5.3 ± 3.5 | 8.6 ± 5.8 | 21.7 ± 5.0 | 12.3 ± 5.8 | 11.4 ± 6.2 | 5.8 ± 4.8 | 4.5 ± 3.3 | <0.0001 |
| L. (V)brasiliensis | 20 | 3.4 ± 3.1 | 8.2 ± 6.2 | 14.9 ± 5.5 | 10.8 ± 4.9 | 5.8 ± 4.2 | 3.2 ± 1.9 | 3.0 ± 1.9 | <0.0001 |

¹Fraction 3 vs other fractions

The results of intradermic reaction-assays for cellular immunity are shown in Table 16. The data indicate that the first-generation immunotherapeutic agent is inducing a TH1 response in cured psoriasis patients. Fraction 3 of the L.(L)chagasi and L.(V)brasiliensis antigenic components of the first-generation immunotherapeutic agent demonstrates the highest immunogenic activity in vivo with the intradermic reaction assay after clinical remission of lesions. Fraction 4 from either of these species also shows a high degree of activity.

### EXAMPLE 16

### Single Blind Trial with Second-generation Immunotherapeutic Agent Containing Isolated Protein Antigenic Fractions

TABLE 17
Response to vaccination with second-generation
immunotherapeutic agent.

| Numbers | | Numbers | | | % Decrease |
|---|---|---|---|---|---|
| of | Frac- | of | Initial | Final | in Final |
| patients | tion | Doses | PASI | PASI | PASI |
| 3 | 1 | 2.0 ± 1.0 | 25.0 ± 13.1 | 10.8 ± 4.6 | 56.8 |
| 7 | 2 | 2.0 ± 1.3 | 24.9 ± 22.4 | 13.1 ± 23.9 | 47.4 |
| 14 | 3 | 2.1 ± 1.1 | 16.1 ± 14.7 | 1.9 ± 2.9 | 88.2 |
| 11 | 4 | 2.3 ± 0.5 | 19.3 ± 15.1 | 2.4 ± 3.8 | 87.6 |
| 8 | 5 | 2.2 ± 0.8 | 28.8 ± 21.3 | 13.5 ± 15.5 | 52.8 |
| 3 | 6 | 2.3 ± 0.6 | 16.7 ± 1.0 | 8.2 ± 6.8 | 50.9 |

The effect of vaccination with the fractions of the second-generation immunotherapeutic agent on PASI values is shown in Table 17. Fractions 3 and 4 show the highest activity for remission of psoriasis. Two doses of immunotherapeutic agent incorporating either of these fractions decrease the PASI by 88% of their initial values in patients before vaccination. These fractions also displayed the highest stimulation indexes in the in vitro blastogenesis experiments and the highest in vivo intradermic reaction (IDR) diameter after vaccination in the patients cured of psoriasis.

### EXAMPLE 17

### Identification and Characterization of Protein Fractions that Induce Clinical Remission of Psoriatic Lesions

Peptide from acrylamide gels were transferred to nitrocellulose papers and analyzed at the ICBR Protein Chemistry CORE Facility at the University of Florida, Gainsville, Fla. HPLC was performed using a Hewlett Packard 1090 HPLC, digestion was performed with Endo-Lys-C, and amino acid analysis was performed using an ABI 494 Protein Sequencer. Amino acid sequence homology was searched using the BLAST program.

TABLE 18

Amino acid sequence of peptides.

| Protein | | | Peptide | | Sequence | Peptide | Homology with |
|---|---|---|---|---|---|---|---|
| fraction | | Band | number | Sequence | ID | length | human proteins |
| 3 | 82 | 2 | 12 | YEDEINK | 1 | 7 | KERATIN TYPE II |
| | | | 16 | AQYEDIAQK | 2 | 9 | KERATIN TYPE II |
| | 80 | 3 | 13 | EIETYHNLLEGGQEDF | 3 | 16 | KERATIN TYPE I |
| | | | | | | | CITOSKELETAL |
| | | | | AQYEDAIQK | 4 | 9 | KERATIN TYPE II |
| | | | 10 | YEDEINK | 1 | 7 | KERATIN TYPE II |
| | 73 | 4 | 10 | YEDEINK | 1 | 7 | KERATIN TYPE II |
| | | | 12 | AEAESLY | 5 | 7 | — |
| | | | 13 | NYSPYYNTIDDL | 6 | 12 | KERATIN TYPE I |
| | | | | | | | CITOSKELETAL |
| 4 | 82 | 2 | 4 | AEAESLYQSK | 7 | 10 | KERATIN TYPE II |
| | | | 9 | ATNAENEFV | 8 | 9 | KERATIN TYPE II |
| | | | 22 | XXYSELNRVIQRLRSI | 9 | 16 | KERATIN TYPE II |
| | 80 | 3 | 18 | EIETYHNLLEGGQEDF | 3 | 16 | KERATIN TYPE I |
| | | | | | | | CITOSKELETAL |
| | | | 9 | YEDEINK | 1 | 7 | KERATIN TYPE II |
| | | | 11 | AQYEDYAQ | 10 | 8 | KERATIN TYPE II |
| | 73 | 4 | 8 | YEDEINNK | 11 | 8 | — |
| | | | 10 | KYEDEINK | 12 | 8 | KERATIN TYPE II |
| | | | 14 | EIEQYLNLLLASYLDF | 13 | 16 | KERATIN TYPE I |
| | | | | | | | CITOSKELETAL |
| | | | 19 | STMQELNSRLASYLDK | 14 | 16 | KERATIN TYPE I |
| | | | | | | | CITOSKELETAL |

Fraction 3 contained three bands after total reduction and alkylation as is known in the art. All but two of the peptide sequences showed homology to Keratin Type I or II human proteins. Fraction 4 showed similar results to fraction 3. This amastigote parasite keratin explains the effect of the immunotherapeutic agents of the present invention on psoriasis patients. Many authors have postulated that psoriasis is a disorder in human keratin from epidermal keratinocytes.

### EXAMPLE 18

### Analysis of Peripheral Blood Lymphocytes with the Flow Cytometer

TABLE 19
Comparison of lymphocyte populations vs. healthy
controls in psoriasis patients before treatment.

| | 0 DOSES | CONTROLS | |
|---|---|---|---|
| | n = 95 | n = 49 | p |
| CD4 | 30.7 ± 12.8 | 40.8 ± 9.6 | <0.0001 |
| CD8 | 20.3 ± 9.3 | 28.4 ± 9.7 | <0.0001 |
| CD8 − CD4 + | 29 ± 9.9 | 38.9 ± 9.9 | <0.0001 |
| CDS | 66.7 ± 9.8 | 73.2 ± 9.8 | <0.0004 |
| CD8 + CD3 + | 13.1 ± 7.3 | 19.5 ± 8.6 | <0.0001 |
| HLA + | 34.4 ± 9.5 | 29.8 ± 11.5 | <0.0150 |
| CD8 + HLA − | 11.9 ± 5.9 | 14.7 ± 7 | <0.0129 |
| lgE | 6.7 ± 3.8 | 4.8 ± 2.2 | <0.0061 |
| lgG | 0.8 ± 0.5 | 1.2 ± 0.6 | <0.0026 |

All psoriasis patients, before treatment with the first-generation immunotherapeutic agent, showed peripheral blood lymphocyte populations significantly lower than normal healthy controls, with the exception of HLA and IgE markers, which were present at elevated levels.

TABLE 20
Comparison of lymphocyte populations vs. healthy controls in psoriasis patients
with different degrees of disease severity following PASI values.

| | PASI 1-9 | p vs CONTROL | PASI 10-20 | p vs CONTROL | PASI 21-65 | p vs CONTROL |
|---|---|---|---|---|---|---|
| | n = 38 | n = 49 | n = 32 | n = 49 | n = 25 | n = 49 |
| CD45 | 98.9 ± 1.4 | 0.1283 | 99.0 ± 0.1 | 0.1 | 98.9 ± 1.2 | 0.1 |
| CD4 | 36.6 ± 9.2 | 0.0353 | 34.7 ± 12.6 | 0.0334 | 22.4 ± 10.2 | <0.0001 |
| CD8 | 23.1 ± 8.6 | 0.0047 | 20.0 ± 9.3 | 0.0008 | 18.0 ± 6.7 | <0.0001 |
| CD8+CD4+ | 2.2 ± 1.5 | 0.6253 | 1.7 ± 1.3 | 0.8163 | 1.6 ± 1.1 | 0.8379 |
| CD8−CD4+ | 36.3 ± 9.7 | 0.1838 | 28.6 ± 10.4 | 0.0014 | 28.1 ± 8.3 | <0.0001 |
| CD3 | 70.8 ± 9.4 | 0.1100 | 66.3 ± 10.9 | 0.0055 | 62.0 ± 9.8 | <0.0001 |
| CD3+CD8− | 57.1 ± 10 | 0.0765 | 51.2 ± 11.6 | 0.9311 | 51.3 ± 7.9 | 0.9802 |
| CD8+CD3+ | 15.5 ± 8.5 | 0.0184 | 14.0 ± 8.5 | 0.0100 | 12.8 ± 6.9 | 0.0030 |
| CD8+CD3− | 6.8 ± 3.6 | 0.4337 | 4.7 ± 2.6 | 0.1182 | 4.4 ± 3.9 | 0.0344 |
| TCR | 2.1 ± 1 | 0.4337 | 2.1 ± 1.7 | 0.3633 | 2.1 ± 0.8 | 0.1441 |
| HLA+ | 32.5 ± 7.9 | 0.3389 | 32.8 ± 7.7 | 0.2202 | 35.8 ± 9.2 | 0.0424 |
| CD8+HLA+ | 8.4 ± 4.9 | 0.0574 | 7.6 ± 5.2 | 0.0418 | 12.8 ± 9.6 | 0.4227 |
| CD8+HLA− | 12.1 ± 4.6 | 0.0483 | 12.6 ± 5.8 | 0.1801 | 9.8 ± 3.7 | 0.0039 |
| CD19 | 7.4 ± 3.6 | 0.8455 | 8.4 ± 4.3 | 0.2806 | 8.0 ± 3.5 | 0.5216 |

Peripheral blood lymphocyte populations were studied in psoriasis patients before treatment with the first-generation immunotherapeutic agent. Patients were distributed according to severity of the disease, tabulated according to PASI values. The results are shown in Table 20. As PASI values increased in psoriasis patients, peripheral blood lymphocyte populations of CD4+, CD8+, CD8−CD4+, CD3, CD8+CD3+, CD8+CD3−, CD8+HLA− decreased while populations of HLA+ increased relative to healthy controls. In the group with PASI 1-9, only four lymphocyte populations were lower than control values, while in the group with PASI 21-65, seven lymphocyte populations were lower than values for healthy controls. This suggests that lymphocytes migrate from peripheral blood to dermis and epidermis in the skin of psoriatic patients to induce the chronic inflammation characteristic of the disease.

TABLE 21
Comparison of lymphocyte populations in psoriasis patients with different degrees of disease severity.

| | PASI | PASI | | | PASI | | |
|---|---|---|---|---|---|---|---|
| | [1-9] | [10-20] | p | I.C. 95% | [>20] | p | I.C. 95% |
| CD4+ | 36.6 ± 9.2 | 30.5 ± 13.9 | <0.4982 | | 22.4 ± 10.2 | <0.0001 | [−19.1 a -9.7] |
| CD8+ | 23.1 ± 8.6 | 23.8 ± 13.5 | <0.1984 | | 18.0 ± 6.7 | <0.039 | [−9.3 a −1.8] |
| CD8+CD4+ | 2.2 ± 1.5 | 2.0 ± 2.1 | <0.2139 | | 1.6 ± 1.1 | <0.0001 | [34.2 a 44.5] |
| CD8−CD4+ | 36.3 ± 9.7 | 26.7 ± 12.1 | <0.0330 | [−14.7 a −0.6] | 23.1 ± 8.3 | <0.0001 | [−20 a −7.5] |
| CD3 | 70.8 ± 9.4 | 67.5 ± 11.5 | <0.0792 | | 62.0 ± 9.8 | <0.0002 | [−15.5 a −4.9] |
| CD3+CD8− | 57.1 ± 10.0 | 50 ± 14.2 | <0.0476 | [−11.9 a 0.05] | 51.3 ± 7.9 | <0.0118 | [−13.9 a −1.8] |
| CD8+HLA− | 12.1 ± 4.6 | 13.0 ± 6.1 | <0.07337 | | 9.8 ± 3.7 | <0.0310 | [−4.4 a −0.21] |
| IGA+ | 5.1 ± 2.9 | 7.4 ± 3.6 | <0.0443 | [0.06 a 4.6] | 10.5 ± 7.0 | <0.0001 | [5.3 a 12.8] |
| IGD+ | 11.5 ± 3.5 | 16.4 ± 9 | <0.0387 | [0.17 a 6.25] | 14.9 ± 6.0 | <0.1462 | |

There are significant differences in lymphocyte populations between patients with different PASI values. Comparison of 1-9 and 10-20 groups shows four lymphocyte populations with lower values in the group with a more severe psoriasis. Comparison between groups with PASI 1-9 and PASI greater than 20 units showed seven lymphocyte populations with lower values in the group with severe psoriatic lesions. IgA+ lymphocytes were higher in the group with more severe disease.

TABLE 22
Comparison of lymphocyte populations vs. healthy controls in
psoriasis patients with total remission of lesions after more
than 10 doses of first-generation immunotherapeutic agent.
Cured patients > 10 DOSES of immunotherapeutic agent

| | | p vs. CONTROL |
|---|---|---|
| | n = 49 | n = 49 |
| CD45 | 99.2 ± 0.4 | 0.1283 |
| CD45 RO | 43.9 ± 7.0 | 0.5406 |
| CD4 | 43.2 ± 9.4 | 0.7561 |
| CD8 | 27.3 ± 6.6 | 0.3985 |
| CD8+CD4+ | 1.4 ± 0.7 | 0.2537 |
| CD8−CD4+ | 40.5 ± 6.6 | 0.9923 |
| CD3 | 70.0 ± 9.5 | 0.063 |
| CD3+CD8− | 51.7 ± 9.2 | 0.5583 |
| CD8+CD3+ | 16.2 ± 5.0 | 0.0634 |
| HLA+ | 39.1 ± 9.6 | 0.0108 |
| CD8HLA+ | 14.9 ± 7.1 | 0.0766 |
| CD8HLA− | 12.4 ± 4.0 | 0.1113 |
| CD19 | 10.9 ± 4.9 | 0.0031 |

After clinical remission of lesions all peripheral blood lymphocyte populations returned to normal values, similar to healthy controls. Only HLA+ and CD19 lymphocyte populations had higher values than normal controls, probably because of lymphocyte stimulation after immunotherapeutic agent treatment.

Psoriasis lesions are induced in skin because T lymphocytes are transferred from the dilated skin capillaries to the dermis. The lymphocyte abundant inflammatory infiltrate induces epidermal proliferation, epidermal thickness, parakeratosis, and scaliness. It is the activity of the lymphocytic infiltrate, consisting primarily of T cells that is the driving force for the induction of the changes in psoriasis, while also being necessary of the maintenance of the plaques.

The process of initiation and maintenance of psoriasis depends on activation of T cells, migration of T cells into the skin and secretion of cytokines by T cells in the skin. T cells must become activated to induce and/or maintain psoriasis since they must be present in the skin.

The process of T cell homing to the skin is regulated by secreted factors and interactions between the T cell and the endothelium. The first step or rolling is mediated by cell-cell interaction between cutaneous lymphocyte antigen (CLA) on the migrating T cell and E-selection on the endothelial cell. This process includes the activation of surface proteins on the T cells mediated by chemokines and T cell endothelial surface protein binding by LFA-1/ICAM and VLA/VCAM interactions completing the T cell migration through the blood vessel, a process called dispedesis.

Finally T cells, local macrophages, dendritic cells, vascular endothelium and even keratinocytes themselves, by a cascade of cytokines secreted by many difference cells, induce the keratinocyte changes in psoriasis.

In addition to psoriasis, other related maladies have a similar mechanism of action. For instance, atopic dermatitis appears to have a similar mechanism of action. Administration of the compounds with the same methodology disclosed herein have shown significant regressions in lesions of patients with atopic dermatitis. Additionally, psoriatic arthritis has a similar mechanism of action. Psoriatic arthritis occurs in approximately 15-20% of psoriatic patients. Psoriatic arthritis effects synovial joints which are composed of two adjacent bony ends each covered with a layer of cartilage, separated by a joint space and surrounded by a synovial membrane and joint capsule. Arthritis is characterized by an inflammatory response of the synovial membrane that is conveyed by a transendothelial influx of lymphoid cells and local activation of a variety of mononuclear cells such as T-cells, B-cells, plasma cells, dendritic cells macrophages and mast cells as well as new vessel formation.

In order to treat any malady that arises from the activity of lymphocytic infiltrate one need not immunosuppress or eliminate T cells, but rather one can provide an immunostimulator, as illustrated by the blastogenic assay reported in Tables 11, 12, 13 and 14. Fractions 3 and 4 had the highest stimulation indexes in human peripheral blood lymphocytes of patient's after 100% remission of psoriatic lesions.

After analysis of lymphocyte populations in peripheral blood with the flow cytometer several lymphocyte populations decreased as PASI values increased in psoriatic patients as shown in Tables 20 and 21, as compared with normal healthy controls as shown in Table 19. After clinical remission of lesions, peripheral blood lymphocytes returned to normal values as shown in Table 22.

Therefore, a treatment for psoriasis and related maladies has a mechanism of action that includes an inhibition or blockade of T cell rolling by interference with the CLA-E selectin interaction by a novel cytokine and interference of endothelial binding or diapadesis by a novel cytokine induced by stimulation of an unknown T cell clone that blocks the LFA-1/ICAM interaction and/or the VLA/VCAM interaction with endothelial cells. Indeed, the first clinical sign seen in patients after the administration of the presently disclosed compositions is the decrease in redness of the skin that is the result of a decrease in the skin capillary vasodilatation typical of psoriasis.

Psoriatic arthritis occurs in approximately 15-20% of psoriatic patients. Rheumatoid arthritis (RA) is a chronic inflammatory and destructive joint disease that affects approximately 0.5-1% of the population of the industrialized world and leads to significant disability and a consequent reduction in the quality of life. RA is a disease in which the immune and inflammatory systems are linked to the destruction of cartilage and bone. The links between the two systems remains elusive, however, and the underlying cause of RA unknown. RA is similar to psoriasis and has a polygenic basis, but the genes involved have not been defined. There is a strong association between RA and several types of autoantibodies. The most important autoantibody is rheumatoid factor (RF), which is directed against the Fc portion of IgG. It has been speculated that RA, as well as psoriasis, could be triggered by infectious agents, but proof of this is still lacking. The reason for the joint-specific localization of the inflammatory response is also unknown.

Like many forms of arthritis, RA is initially characterized by an inflammatory response of the synovial membrane (synovitis) that is conveyed by a transendothelial influx and local activation of a variety of mononuclear cells, such as T cells, B cells, plasma cells, dendritic cells, macrophages, mast cells, as well as new vessel formations. There is a strong association with the mechanisms that lead to homing of involved cells to the joint and subsequently trigger a T cell response.

The synovial joint is composed of two adjacent bony ends each covered with a layer of cartilage, separated by a joint space and surrounded by the synovial membrane and joint capsule. The synovial membrane is normally less than 100μ. The T cells infiltrating the synovial membrane are primarily CD4+ memory cells similar to the T cells found in skin of psoriatic patients. The synovial membrane is normally less than 100 μm thick and the synovial lining, facing the cartilage and bone, consists of a thin layer of synoviocytes, with one type derived from macrophages and the other type from fibroblasts. There is no basement membrane. Only a few mononuclear cells (if any) may be found in the sub-lining connective tissue layer, which has considerable vascularity. The synovial membrane covers all intra-articular structures except for cartilage and small areas of exposed bone and inserts near the cartilage-bone junction.

The lymphoid infiltrate can be diff-use or may form lymphoid-follicle like structures. This is process is similar to the inflammatory process in the psoriatic skin. The lining synovial layer divides continuously, become hyperplastic, with a thickness greater than 20 cells (i.e., >100 μm, and subsequently the synovial membrane expands and forms villi. In addition, there is bone destruction. This process may also be seen in psoriatic arthritis. As a result, treatment with the polypeptides of the present invention may halt the traffic of lymphoid cells from the blood to the skin, and also from the blood to the synovial membrane, thereby acting to reverse the inflammatory process that leads to chronic inflammation in both RA and psoriatic arthritis. By immunostimulating the T cells that produce the novel cytokines that inhibit the vascular process on the T cell receptor or on the Endothelial cell receptor, the polypeptides of the present invention may stop the traffic of lymphoid cells.

The foregoing description of specific embodiments is merely illustrative, and various modifications may be made without deviating from the spirit and scope of the present invention, which is limited only by the following claims.

What is claimed is:

1. A method for selectively inhibiting T-cell rolling in a human host, comprising administering a compound that selectively interfers with the CLA-E selectin interaction and LFA-1/ICAM and VLA/VACM interactions.

2. The method of claim 1 wherein said compound is an immunostimulant.

3. The method of claim 1 wherein said compound includes an immunotherapeutic agent, said agent comprising a purified protein extract wherein said purified extract is isolated by diethylaminoethyl Sephadex chromatography of a Nonidet P-40 insoluble particulate antigen fraction derived from isolated killed cells of amastigotes from at least one species of the Leishmania genus, said particulate antigent fraction solubilized with 8 M urea and 0.025 M. Tris[hydroxymethyl]aminomethane pH 8.3 applied to diethylaminoethyl Sephadex and eluted with a solution comprising 0.1 M. sodium chloride, 8 M urea and 0.025 M. Tris[hydroxymethyl]aminomethane pH 8.3, said purified protein extract including polypeptides having apparent molecular weights after total reduction and alkylation of 73, 80 and 82 kDa.

4. The method of claim 3 wherein the species is Leishmania amazonensis.

5. The method of claim 3, wherein the species is Leishmania venezuelensis.

6. The method of claim 3, wherein the species is Leishmania brasiliensis.

7. The method of claim 3, wherein the species is Leishmania chagasi.

8. The method of claim 3, wherein the species are Leishmania amazonensis, Leishmania venezuelensis, Leishmania brasiliensis and Leishmania chagasi.

9. The method of claim 3, wherein the 73 kDa polypeptide comprises the amino acid sequences set forth in SEQ ID NOS: 1, 5 and 6, wherein the 80 kDa polypeptide comprises the amino acids sequences set forth in SEQ ID NOS: 1, 3 and 4 and wherein the 82 kDa polypeptide comprises the amino acids sequences set forth in SEQ ID NOS: 1 and 2.

10. The method of any one of claims 3-9 further comprising an adjuvant.

11. The method of claim 10, wherein the adjuvant is alumina.

12. The method of claim 1 wherein said compound includes an immunotherapeutic agent, said agent comprising an immunotherapeutic agent, said agent comprising a purified protein extract wherein said purified extract is isolated by diethylaminoethyl Sephadex chromatography of a Nonidet P-40 insoluble particulate antigen fraction derived from isolated killed cells of amastigotes from at least one species of the Leishmania genus, said particulate antigent fraction solubilized with 8 M urea and 0.025 M. Tris[hydroxymethyl]aminomethane pH 8.3 applied to diethylaminoethyl Sephadex and eluted with a solution comprising 0.15 M. sodium chloride, 8 M urea and 0.025 M. Tris[hydroxymethyl]aminomethane pH 8.3, said purified protein extract including polypeptide having apparent molecular weights after total reduction and alkylation of 73, 80 and 82 kDa.

13. The method of claim 12, wherein the species is Leishmania amazonensis.

14. The method of claim 12, wherein the species is Leishmania venezuelensis.

15. The method of claim 12, wherein the species is Leishmania brasiliensis.

16. The method of claim 12, wherein the species is Leishmania chagasi.

17. The method of claim 12, wherein the species are Leishmania amazonensis, Leishmania venezuelensis, Leishmania brasiliensis and Leishmania chagasi.

18. The method of claim 12, wherein the 73 kDa polypeptide comprises the amino acid sequences set forth in SEQ ID NOS: 12, 13 and 14, wherein the 80 kDa polypeptide comprises the amino acids sequences set forth in SEQ ID NOS: 1, 3 and 10 and wherein the 82 kDa polypeptide comprises the amino acids sequences set forth in SEQ ID NOS: 7, 8 and 9.

19. The method of any one of claims 12-18 further comprising an adjuvant.

20. The method of claim 19, wherein the adjuvant is alumina.
